SEQUENCE LISTING (1 sequence)

SEQ ID NO: 1 - DNA, 61696 bp, Unknown
  misc_feature: Bacteriophage RKP180
  misc_feature (1)..(201): ORF1 hypothetical protein
  misc_feature (191)..(1393): ORF2 tyrosine recombinase
  misc_feature (1695)..(1964): ORF3 pyocin activator protein PrtN
  misc_feature (1976)..(3238): ORF4 hypothetical protein
  misc_feature (3278)..(3514): ORF5 DNA-binding protein
  misc_feature (3511)..(4467): ORF6 RecT-like protein
  misc_feature (4509)..(5537): ORF7 RecB-like nuclease
  misc_feature (5729)..(6208): ORF8 Fis family transcriptional regulator
  misc_feature (6209)..(6799): ORF9 transcriptional regulator
  misc_feature (6928)..(7296): ORF10 transcriptional regulator
  misc_feature (7302)..(7388): tRNA-Ser
  misc_feature (7453)..(7980): ORF11 single-stranded DNA-binding protein
  misc_feature (7994)..(8218): ORF12 hypothetical protein
  misc_feature (8208)..(8693): ORF13 hypothetical protein BcepIL02_gp15
  misc_feature (8693)..(9073): ORF14 DUF1364 family protein
  misc_feature (9236)..(9466): ORF15 hypothetical protein
  misc_feature (9463)..(9915): ORF16 hypothetical protein
  misc_feature (10863)..(11387): ORF17 replication protein 15
  misc_feature (11374)..(12030): ORF18 hypothetical protein
  misc_feature (12088)..(12525): ORF19 hypothetical protein
  misc_feature (12522)..(12821): ORF20 hypothetical protein
  misc_feature (12818)..(13177): ORF21 hypothetical protein
  misc_feature (13203)..(13361): ORF22 hypothetical protein
  misc_feature (13358)..(13837): ORF23 IQ calmodulin-binding domain-containing family protein
  misc_feature (14402)..(14830): ORF24 hypothetical protein
  misc_feature (15188)..(15826): ORF25 terminase small subunit
  misc_feature (16080)..(16283): ORF26 hypothetical protein
  misc_feature (16323)..(16631): ORF27 hypothetical protein
  misc_feature (16699)..(16857): ORF28 hypothetical protein
  misc_feature (16854)..(16997): ORF29 hypothetical protein
  misc_feature (16994)..(17344): ORF30 hypothetical protein
  misc_feature (17356)..(17577): ORF31 transcriptional regulator
  misc_feature (17558)..(18319): ORF32 hypothetical protein
  misc_feature (18316)..(18987): ORF33 hypothetical protein
  misc_feature (19008)..(19712): ORF34 hypothetical protein
  misc_feature (19709)..(19912): ORF35 hypothetical protein
  misc_feature (20219)..(20434): ORF36 hypothetical protein
  misc_feature (20431)..(21027): ORF37 hypothetical protein
  misc_feature (21024)..(21224): ORF 38 hypothetical protein
  misc_feature (21200)..(21451): ORF39 hypothetical protein
  misc_feature (21549)..(21740): ORF40 CoA ester lyase
  misc_feature (22825)..(24147): ORF41 terminase large subunit
  misc_feature (24144)..(24569): ORF42 hypothetical protein
  misc_feature (24586)..(26931): ORF43 portal protein
  misc_feature (27006)..(27296): ORF44 hypothetical protein
  misc_feature (27433)..(27882): ORF45 hypothetical protein
  misc_feature (27892)..(28341): ORF46 hypothetical protein
  misc_feature (28351)..(28548): ORF47 CsrA-like transcriptional regulator
  misc_feature (28624)..(28911): ORF48 hypothetical protein
  misc_feature (29062)..(30171): ORF49 Major capsid protein
  misc_feature (30249)..(30722): ORF50 Virion associated protein
  misc_feature (30779)..(31318): ORF51 hypothetical protein
  misc_feature (31322)..(31984): ORF52 Virion associated protein
  misc_feature (31981)..(32601): ORF53 hypothetical protein
  misc_feature (32611)..(33033): ORF54 Virion-associated phage protein
  misc_feature (33040)..(34017): ORF55 Virion-associated phage protein
  misc_feature (34014)..(35720): ORF56 minor tail protein
  misc_feature (35720)..(36001): ORF57 Virion-associated phage protein
  misc_feature (36001)..(37320): ORF58 putative tail fiber protein
  misc_feature (37406)..(38278): ORF59 tail fiber protein
  misc_feature (38290)..(40023): ORF60 Virion-associated phage protein
  misc_feature (40023)..(40343): ORF61 Virion-associated phage protein
  misc_feature (40419)..(40865): ORF62 Acyl-CoA N-acyltransferase
  misc_feature (40855)..(41688): ORF63 Virion-associated phage protein
  misc_feature (41699)..(42424): ORF64 PAPS reductase-like protein
  misc_feature (42421)..(43950): ORF65 Virion-associated phage protein
  misc_feature (44008)..(57543): ORF66 lytic transglycosylase domain-containing DarB protein
  misc_feature (58023)..(58244): ORF67 hypothetical protein
  misc_feature (58287)..(58598): ORF68 hypothetical protein
  misc_feature (58762)..(59355): ORF69 DUF3380 domain-containing N-acetylmuramidase (lysozyme-like) family protein(endolysin)
  misc_feature (59321)..(59968): ORF70 hypothetical protein ADU18_0048
  misc_feature (60035)..(60262): ORF71  hypothetical protein
  misc_feature (60355)..(60735): ORF72 chemotaxis protein
  misc_feature (60695)..(61114): ORF73 lipoprotein
  misc_feature (61114)..(61647): ORF74 Rz-like phage lysis protein
  misc_feature (61352)..(61597): ORF75 Rz1
atgcctcaatatgacttggatgcaatggatcgcgggcaattggtggaggagcgcccgatcacgtggaaagcgcggattttattcgccgtcgcggcggtcgtggtgtacgcggccgtcagccatttcgggcagacggccatcgtggcgatcggcgcggcgacggtcggagccatcgccggcatgttcttcctcaagcgctgatcctgcgttctctgcgggccgctgcctgggcagcaagggcggcgctgttgtcgcccggcccacggcgcttcagctcgatcccccgccggcgcggccgggccggctcgatgccgatggcaatgtcgagccacttccagccgtcgtagatgtcgccggtccccttcagcctcgtgtagcggcgcagcacgttccagtttttgtgcgcgcttaccctcgtcacctgcgggatgtccagtcccgtctcgaacaggtgcgacacgccctcgcggcgcaggtcgtgaaagcgcaggtcttcgataccgagccaagtgcaggcgcgctcgaatgtcgcagacaccgcgtcggtggtgtaggggaaaatccgctcctctatctgcggcatggagtgcagcaggcgccatgcgcgatcgggcaggtagcacaacacatcgttgcccttcttcttcgtcgggtgcttcatgtcgcgcaccaggatggattgctcctccgcattgatgtcatcccagcgcagtttcacgatctcctcctggcgccggcaggagaagatggcgaagggcgcgatcttgaccagtggggtgatgtaggcccgccggcggatcgcctcgtggaagtgttgcatcagccggtcgagttcgtcgcgcgtcggccgccggtcgcgctcctccgggcgctcggtcaggcccatgtgccggcacaccttgcgcgcgtccgcgatggcgtttgggtcgagctgatagccccatgccggcctcgccagatcgaagacggtgcccagcagggacatgtcgctgttgacggtctgcggcagcacgcagtccgtggcgatgcggtcccgcgcgtagtcgaccagcgtggcgctggtgataatcgaccccttgcactggccgagttccgacttggcgatggcgcgcagggtgccctcctgggtgcgccctaggccgcgccgttcggccacctcatccaggaagcgctcgatgatcttgcccagggtggggtcgggtgccgacatcgcctcaatggcgcccggctctgccagtgccgtctcgcgcttcttgacccatgctttggcggcggcctctctgtcgaatgtctccgtctctttatgaacgacggcgccatcgcgcatgatcctgactcgtgccatataggcgatcgagccgtctttccgtttgcgtgggacaatcgttcccatttgtcacatttccttggttggcattggaccgacaggccatgtaccaaaaaatgtgacagcggccacttaaaaacgggcttgcaagggcaagaatggtgtggaaatcgtaccgattaaggcgaaaagcgtaccgctagaaatgggtaaaagcgcagtgaaatcaacggtatgcgatagtcataagttctgcatcgcaccgatgatggattggaccgacaaccggcttttgtattggaccgacaggcacttgccgttgtgacatcgccagatgtggcgaatttgtgccagccgatacgccggctcagagccggcgcccatgcagttgctcaaactcctttgtggcttcggcccgctggcggtcgatgtagtcggcaaggtcttgcaggtgcacgccgcgcgccgacttctgcgagcgctcgatgcgcatgatggggagggggatgtcgcccgccgcgaccttgcgggcgaacttgtcgggcgtcaggtgcgcgaagaaatcgcggcacacgtcgtcgagcggtatgacggccttcccgccgtattgtgcgagcagcaggaaggccgtcctcattgacatgccgctcactggccgccgtccaggctggagcggaacaggtcggcggcgtcctccggcgtggccggcgtcgtgatggcgttgccgttgccgtcgccggcgaccacgaacgtgacgtcgcggccatgggtccagaacggcgtcagcttcagctcctggccgatgaactcggccgccttgccgttgccgtccggcgtcgtgccccaatactcgccgttgcaccagtagccggccggcgccatgtcctcgggcggcatggccggcgcatccacgatcgtaatgacggagccgctgcggtgggccatggcgatcacggtttcatcgccgccgcccatgtcgacgaacgcccgtacttcaggcgaggcgtctggcgcgcacgtgatgacgacctgggcgccgctctcatcgacgccagcggcggcaaacaggtcgttcacgtccaccacgcccggagcgactgccgcgagtgcgcctgccgtgctgatcgccagcgccttctcctgcatggcctcctgctggcgcgcgatcgcggcctgttgctcggccaattcgcgctgctggcgctccagctccgcgcgttgctcggccatgcggcgagcttcctcctcctgggcggcgcgctcggcggcgagccgtgcggcttccgcctcgcgggtcttggccgactccaggagcgtgctcaggatgcccagggcgttgtcgcgcgccagtgtggcctcggccacgaactcggcgaaaacatcctcggtgggcgtgaagccttgcaggtcgtcataggtggcttgcagttgctcggcggtgtcgctggccgactgcattggcagtgcccggatgtcgttgatcttggcctggatgcccgccacgcgctcacgttcggcgcgtgcgcgctcctccttttccgcctcgatctttgcatcgtagtcgtcgcgcaggccgaatattcgttcctcctccgcctcggtgatgccgagcaggcgcttctcctcggcgatcacggcggagcagaacgccgtggcatcctcgcgcgcggccttgccagtcttggtgatggtgatgcgggcattcttcaggttcatgcctgcgcggtgcgcttcttcgcgtccgttagcgtcgaccacggtggtgatcgcggccgacttggcgaccagctcgcgcaggtgcgtctcggtcttggtggagtccagcacgatcacggcacgctcggccggcggcaggctggagatggtcgtcacggccgtttgctcggcggcctgggcgttcttcttggtcatggtagtcccttgtgctgtggtggtggagaaagggattggtcaggcggcagcgttctcgccggccgcgtcagtggcgccagccggcgccttgccgccctggaggcagcgctcctcgtattgcctgatgtcttcttcgcggtagcggatggtgttcttgccgatcacgatccaggcggggccggtcttctgccggcgccacttctggagcgtgcgctccgtcacgccgaagcgcttggcaaggtcggtttcggtcaggtggtggcccatgcccgtcactccgctgcctgcgcctgggcctgggcgtcggtggccggttggcgcacggcttcctgcttggccttgttgacgatgccgccgaagcgggaagggcggcgcgcttcctgttcggcgcggccgtccgtcagggccgccggcgccgcgcccgccactgccgccgtggcgcccaggtcgagcacttcgccgcgctggttgaagccgtcgtagtcctcgttgtcgtggccgatcgcgctctccaggcgcttcgagctgttgggcagcaacttggcggcgcgcttgatgacggacttgatcgccatctggtcgtaccacttcgtccacgggctgttggcgccgtcgccggacttggacgcgccgcgcaccttctcgatgtcacggcgcggcatgacctcgcggtgcacttcgccgttggtgaagcgcacgacgatgtaggccgcgacgatctttccggggtcgtcctcgcccaggtacggctcgtgctccaggcgcggatcgtcgccgcgcacgaaccggaactggtcgcgctcgtacacggcggcggcgtcgacgtggctgatctcgccggagtttcggatcaacttcagcaggccgcgcaccatgggcaggtactgaacggtcggcacccacttatcgacctgccgattgccttcccacaccttggtcttggtggagtagacgttcagcactgcctcgcgggcgtccggcaggaggccgtcctgtgcggccttcatgcaggcgacgaacagggattggcggtcagcgtcgagcagatcgtggttcacctgcacggcggtcagtgccgtgcggatgaatcgctccgccgggatatcctccggcagggccttggcgatctcgcccttctgcttctccagggtgttgcggatgtgggcaagctcctgccggcgccaatcggcttcggtcatcggctgctgctgttggttcgggtcggtcatgatgatcgctcactcggtatggtgcgggatggtgcggcagctcaagccgccttgtccggggtaatcctgatgttgcgaaacggcgccaccgtggcctcgatcgtcgtggccgggatgtcgctcacgctgatggtgtagcgcttgctggcagcgcgatcgtagacgcggtagctctccttgttggtgccggccgagatgttgaagccatcggcgcggatgctcttggcgtgctcgatgatcgcgatgatctcggcctgcgccgccttcttccggccctcggccgctttctcctcggcgccggcggccttgtacgctgcacacagctcggcaaggcgcggctcgtcggacatgtcgacgctgctgccgtcgttgtcgcggtagagctgcttgatcgtgtcgccatcgcgcgtgtagtccggctgcggcgcggcgccggcgtcgacgcgcatccagaactgcgccaccttctcgcggatcagcgcgccgacttctgtgtcgcgttcgcgcaacgagaccttcggcgtgttgccgccgaccagcggggcgatcatcgaccagcccaggcccgcgacttcaagttggtgctgcacctggaattcgatgtgcgccggcgcctcgatgtcgtcgccgtcgtcaatccaggcgcggcggaattgcaggccgtcgacgttcttgatctccatgatgccgtcgccgtggcgctcgaacatgcggcgggcttcgttggacgggtggccggcaaccgtgccgacgatcttgaagtcgaacgacgcgcccatgcgcagcgccgggaggcgcatgtacgccttgaacggttcgacgatcaggccgtagtcctcggcgatgccggcggcgatggcgctttccaggcgcttgccccaggtcatccgctcgttctccttgaactccgccacgatgcgcttggtcttgcgcatgaacagctcgtattcggtggcgtagggcgaggcgccgaacagggcggcggcctcggtgctggtcaggtcgtcggcccgcatcgccagccattcggcctcgttggcggggttcagcgtcacgcgttcaatgctcatagtatgatctccatgcgccaccctatgcggcgtgcatgcagaatagcaccagtggtacgaaaatcaaaccgtttttcttcaatgttcgcaaaaagcgtcagcagttgcgtgagagctacatccaacggcatgcgcgagatggagcatgaagttagaatttgcacctatcgcaaactcatccacgtcgaatcatggagctgacacaagaaaggctgaaggagcttctgcgctacaacccggagaccggcgagttcgcgtggttggtcgaccattgtcgcgtcaggatcggttgccgagcggggacaaaggcgaaagatgactacgtgcacatcacagtggacaagcgcagaaccttggctcacaggctcgcatggctatacatgactggccgctggccgaatggaataatcgaccacgccaacctcaacaaaagtgacaacaggttttgcaatcttcgagaggcgacgttccagcagaacaacgtcaacagaaaagccaaagcggcgtccggctacaagggagtgtgccgagttgggagccgatggaaggctatgatatgggctggcggcaccagccgacatcttggctactttgactccgcagaggatgctcacctcgcctatgttgaggcatccaagaagacccacggagaattttctagagcggcgtagttacggcctgatcatgcgcaccggtgccgcgctttctagctttacgttttccttggtgaagaagccagaaaggttgtatgcgccgtcgcggtagccacgccgcaccgtggcgatcacttcatcaccgtcctttatcttgacgtggcagaggcggcctatcgcatctggatgaatggtatcggacggtgcatagaacaatagccagccgtctgcccacgagagcggtgtgtcggccgtgcggcactgaattgcctcagtgccatccggcgacccaggcggcgccgacgtgcgctcgacgtgcttccccgagatcttctcgacgtggccgtcgccgcgcagcgcgccgacgacagacacgcgcgagctgctgatcgggcgggccgatacgcccgcgttctcgacgatctcgtggatcggcacaccgaagatgttggagagctgcgcggcttcctccaactgcatgcgccgcgagccgctgaatgtgatgctgagtgcggagtgggcgaggcccatcttctctgcgagctgacgcagagagattttccgctgtgccatcaggccctcgaaataagagcggtttacttctgtcataccctgtagccggttttcattgcggttcgcatacaacaccactcacacaaaaaagcaaccacgcggcttgccggaatggtacgatttcattaccatcatcatagacgattctctaaaaggatagcgatatgaatatgttgaccgagctggacgtcaccggaccatcagcccgtgcgctgcgcaaatcgctggggatgaagcagactgagttctggaaccgcatcggcctgtcgcaatcctgtggttgccgctacgagcgcgagggtagcccgattccccttccgtaccagcgcctgatcttcctgcactacgtggccggcctgccggtcgatgccggcaccaaggagaaggccgcaagcctgatcgagctgggcacgatcgccaaggacgccgacaaggttacgcgcctgcgcgctgcggcggcgaaggccgacagcatttcggctgcgcttaagggcgcggtcggtgccgcgagcgagatcagcgcggccgtttgatccatggcgggttgggtgagtggcttaaacctgcgttcttgaaaagcgccgagccggcaacggctccatccgttcgaatcggatacccgccaccaatttccacgccgccagcaaggcggctttcgcactaatcagcaccataccaaagaggcgagcatggcatccgtcaataaagtgatcatcgtcggcaacctgggggccgatcccgagacccgctacatgccgagcggcgacgccgtgaccaacatccgcgtggcgaccaccgaccgctacaaggacaaggcaagcggcgagatgaaggaatccaccgagtggcaccgcatcgcgttcttcggccgcttggccgagatcgccggcgagtacctgcgcaaaggatcgccggtctacgtcgagggccgcctgaaaacgcgctcctgggagaaggacggccagacgcactacagcaccgagatcgttgccgagaacatgcagctcctcggcggccggcaggacgacggcggcggccagcggcagcgcggcggtcagcagcagcagcagcgcggcgcgcagcagcaggggcagcagcgcggcagccagcgcaacgagtacgccgagcagaccgggcgcggcaaccgcacgccccaacatcgtcagccgccgccgtccaacggcttcgaggacatggacgatgatatcccgttctgacgaggcgccgaccatgagcaaccacgctgacattctcgacgacgctgccgacctgacgcagcgcgagaccgattcactggtggctgccgcgcgcgagcgtgcggcaaagccgatcccgacagcgacgcactgcatctattgcggcgaggagactgaagacgggcgccgcttctgcgacaagctgtgtctcgacgcgttcgagcacgagatggctgcgaggaagcgcaatggacgctgagatcaagccgacattccagggcgaagtgcagcttgcgggctggtctgagacgcacaacggcgggtgcaaggtaacgttctggctggcggacgccaccgacctggacgtgttccgcgcactcactgtgcgcaagggcaacacggccggccatcgcctcgcatgtgttctcgtcgagatcggggaagatgagcttccggtcacgccgccggagccggtcgcacccgcgcctgagaagccgcagggcggtgcgctggcgaagctcgccggcatgctgtgctctaactcggatttctggcgtttccttacagagcacttccgcgcgtcgatcaccaacgcagagcaggccgctggagctgtgcgccacatctgcaagatcgagagccgtgccgagctggacaacaacgcggacgcggccagcatcttccacgaggcaattcgtctgccgtttgtgcggtggcagcagggggtgcgctgatgcttcgacgcaagacgccgcttgcgcgcggcacgtcgcagctccggcgcagcgccatcaagaagcgtgcaccgaagaagcggcccggccatgacaagcgctaccgcgacgcgtgccgcgaccagcactgctatctcattgtgccgggtgtgtgctgcggcgacaccgagacgacggtgccgtgtcatccgaactggagcgactacgggaagggcgccggcctgaaggcgccggacaagttcaccgttccgggttgctggcgctgccatgcgtggctcgatcaaggcccagcgccgcgagacgagaagcgtcaggtgttcgagcggggtcttgctcgttggtccgcataccgtgagcagcgccttgggctggccgcgtgatgcgatttgcataaacacaacaacacgaacgaaaggggtgatggcacaatgtcattgccaaccaaaaattcgagtagccgtaggacaggaggcagcccgttactcgacgggttggttggcgccattgcctcctgtcctacggctttttcatttggagtacgtatggatgcgaagtctcaaaacgtcgttgtcctcgaccacctgaaaaaggtcggccccatcacccctctggaggccctgcgcctctacggcatcatgcgcctcggcgcccgcatctacgatctccgtgggagcggccatctcatcatcaccacccccgtccgtctgaaagcgcgcaagggcgccaagcccaaggtcgtggcgagctactctctgacgaaggcggcggcatgaggcgccgcagcccgatcagcaccggcgccgtgcgccgcgcgccgcgccgcagtcccgagttcgacgagcaatgtgcgctgttcgagtgggcgcgcatcccggccgtcgtcgcgcaataccccggcatcgacctgctggaaggcagcatgaacggcgtgaagctgtccaaggcccaggccggcaaggccaaggccgccggcatgctgaagggcgcgcacgacgtgcgcctgccggtgccgcgcggcggatacatcgggctgtctatcgagatgaaggccggcaagaacacgccgaccgatgagcaactgtggtacggcggccggctggatgcggaaggctggtgcgtcttctactgctggagctggatcgacgcgaaggaggcgatcatcgcctacctgggcgggctttcgcttgcgcccagcggtacgaaaatcaaaccggcggtataaaaaaagaggcccgccgaagcgggccgcacagagggacgtgcagagtgatcaagcgaattgtaacttggaaaatgcgcagttggcaataaagcaataccagtggtgcgatttacgtaccgcgagccgcgaatcgtggcaaaatgcagtatgccgcgcagttagcgcggaccccttggccggggttttatcaagtagagcttcacatgcgcggtggcgggcacttgacccgttcggccaacccggaaacgggaccgcgcaggtgaagctcttttttttggagctaccatgaagcatgacccctcaccattgagcttgcggtcgctctgatcgctttcgcgcgttctgccaaccgagcatcgattgagatgcatgattgggcgatctctctcgttgagcgcctgattgaggagagcgatcagaaggatttgctctggcaggccatagtcgacggcccgctgttttcgagcgcgcggagacgcttgctcgccaaactattctctctgcatgggtgacgagtaatggcgcgcatcagaactgttaagccggcgttgttccggcatgaagagctgttcgaccttgagcgtgaggctggcctccctctccggctggcattcatcgggttgttcacgtgttgcgatcgagatggaagattcaagtggtcgccaagaacactcaagctcgacgtcctccctcacgacgatatcgatttcgcacgcgtgctcgacgcgttggcgtcgcgtggtttcgtgcggaagtatcgcgtggatggggaggatttcggtgtcataccgacgttcgcaaggcatcaggtcatcaacaacaaggaaagccagtctgagcttccagagccagaagaaagttcttacataacaataacttgcacgcgtgaggcacgcgtgaacatcgcgaacgcttcttcgctttttctttcccaaggggaagggaatatggaaggggaagggaatatggaaggggaagggaatggttgcgccggggtcgcgccttcggcggaccgaccgcagcgcaccgaaaagccagtcgagttgcttactgcaaaacctacgaaggcgagcacgacgaaaccgcccgcgccaacatcggcgatctgggacgcctacgcggcagcgtatcgccagcgctatggcgcggagcctgttcgcaacgccaaggtcaacggccagcttgcgcaactgctgactcgcctgggcgcggacgaggcgccacaggtggcggcgttctacgtctggcacaacaaccggtactacgcgcagaagatgcacgccgtggactgcttgctggcggacgccgagaagctgcgaacggaatggttcacgggccggcgcatgacggcgaaggccgccagcgaggccgaccgcttgcaggaggacggcgagatgtggggccgaattgcggagattcatgccacccgtgatgcagaggcgttgagaggaggcgatcatggcagggattgatctcatcaaggcggttgccgccacggccgagctgtgcggcgcgcggctgaccgaggcggcggcgatgatgctggtcgacgacctgtcggcgtaccccgaggcggccgtgctgcgcgcgctgtcgaaggtgcgcaagtcgagggcgcgcttctcgctcggcgcgatcctggagcacatcgatgcgatggacgggcgacctgggccggacgaggcgtgggcaatgctgccgaagtccgagcgcgacaccgtggtgtggaccgaggagatgtcgcaggcgtggggcgtcgcggcgccactcgttgaggccggcgacaagttcggcgcgcggcgcgcgttcgtcgaggcgtatgagcggcttatcgaggaggcgcgcgatgccggccgcccgccgcagtgggtcgcgtcgctcggagacgacaagcacggccgcgagccggtgatcgccgctgcggttcagcgtggcctgctgtcggccgggcaggcgcagatgctgttgccgggcgccgacatcgcctccagccccgttgctgcgctgctgaatggtggcaacgccgcgccgctaattgccgcgcagccggcggcaaacagggatgccgcacgcgagcacctgaagcgcttggcgagcgcgctcaagaaggcgtgaaatattccttgtgcattttctgcgagtggtactagaatcgtaccgcagcacctttttatgaccgatgcattttttcagggactgatgatggaccacacgaacaacggcggcccggcgtttccgtcgccggctgagcacttcagcgacggcacactatccgggcacgctacatacggcatgtccctccgcgattacttcatcgcgcacgcgccggccgagccgcagccgtggttcaagccggccatgccggaagcccggcctgagtccccgcgcgcacgcgacgtcctcaacgaggacgacctgcgggactggttcagcgatcggctggattccgagccggaggcatgcagtcccgcccttcgggagttcagcgagcgtctgagcgcggcggcgggtggcgcccgtgcatgggaccaggagcatgaaaagcagcgctacatccagtggccggccgcctgggccgacgagcaactgcgcgcacgggaggcagcatgaacaccgagccggtaatcatcaaggccgcgaagaagcgtcacgcctgcacgtggtgcggcgaagcgatccagccgggggaatcgtacgtgcgatggcgctggttctatgacggcgaggcgggcacgaacaagatgcaccctgagtgcgacaaagcgtgtgagcagatgtcgcgggaggagggctacggcttcgagtggacgccgggcgagttcaagcgcggatgcacgtgtgacgcaggagacccgccgtgcgaccacactgccgtgccggcactgcaagcgaaggaggcggcatgaccttcaatcgtgatgtcaactgctaccgcgcgacgccgcgctcgctgcaaaccagcaagttcggctgctacgcgactctgagcacgacgcgccgcaccatcctcggcacctgggcctgctacgcggtcgcggtgctctcgattgtggccgccaccgtggcgctgtccgggtgcgggccgagcggtgaggcggcgccggaacgcgagcttgtcgcggcaatcgataccgagaccggatgccaatacatccgcgtgtccggcctcggcggcatcacgccgcgcctggacagcgacggccggccgatgtgcggcaaggccagcaagaccaaggcagtcaccctcgttaccgcgcgctgacgcgcaccaacccaggagattcatcatgacagtgatccgcaacaagggcgtcgtgtacgccggcaaggtgggcgcgaagatgcgcccgcgcttcagcagcgcgccgcgcaagggcgggcgctacgtcagcgacgtccccatcggcatcaccaaaccccttttggaatgggagccggcagcatgacgaccgcacagaaccaggccctcggtgtggccgaggccaaggccatgaacaaggccaccaaggagttcacgatcgtcatcatggtcgaccacgagcgcgccgcgcgcatctaccgacaggcgctcaccaagatcgggctgatgatggacgagctgagccgcgcggccgagttcttcgataagcgggcgatcgatgcgcacctgtccgcgcgcgcggcgtgggaggatgacgtggccgccggcgacgatgcagcggtcgagccggcgaagcccaagctgcgctggaacgtgatccagaaccggatcgcgtatgtgaagcgcatggacaccctcgcgcagttcgccgtggcgccgttcgagatgaccgaggggcaggtgtaccagctccatcgcatggagatgggcgactacttcgagccgttcgagacgctgctgaaggactggtgggcggagcgcggcgtcggcgcaacaacgtagctgaaatccttgtggcgcaagggtttcgcgcctacaatggaatctctttcgagccgcgcccacacaggctctgataccgtgggtaacgtgcgagcagccggccaactaggcgctgacagcccggaatagcacgggcaccacccgcaaagcgtccgcgctgggcattttgcgagtggggttgttcgctggcttacagacgactcaaaacgtatccatcaagccggggttgattaccgtcacaatctgacggcctggaaagacaggtaaccggaccacactccggcgagtggctcccgtaaggagccgcctcacgcattgcgattgaacgcgcggcagcgtcccgtgatgggcctaaatgcccgcacaccaaatcgggccagtcgcaagccgtgagggaatggacagtctgccgtccgcccgagcttgtggcagccgccagggcttcattgcaccgcgctttagcagggcaccctcaacgcctcgtcgatagcccgagaacgtaccccactcacacggtgatgagttccagcgcctgactggaatcgaacagaggggatcaggctcctattcggactgcgccgtctgctgggctgtgaaggtttccgcgagcacccggtacatctcccacgggctgggcgccaggatggcgatggcgacgaccacggcatagaccacgctccggctcctcggattctcaagcacgcaggggagccgacgaggcatcaggggcgcgggccggggggcgcgccagcatctccaggccatgatgtgcgaggcatcgccatcatcgtagacgcatcccatcatgatcgcgtctgctggcacgatcaccccgatcgactccaggccgactgacacgaggtcgcgcttgcgcggacagtccatgttgagcagccgggcgtgcgctgggaacgtggccgggcgcggaacgatctccatggactctatgcgatccaccccgttctccagccgccgcaccacgaggtgcatcatgaagtccgtgagcgctgtgtgcccgcgtgagaacgatgaagatggaaccctttcgcacattgctgccttgcccctgcctgcctgtttgccgtgagtcgccggcgtgatacgatttccgtaccacaaccgggatattcgcaccacgtgttgcttattgttcccattagcgagtaccggagttctacgcaacggaatgccgatcgttcgcacaccaacgaaagtgtgagaaacgcgacaaacccttgtgcagagcagggattccgaaatgcatagctgttccgccgggcgcggatagctgtggaaaatcggcaggttggcaacaggcactgagattcgacattgaccaggagagacgagatgtcgcagggcgcatcaacgaagaagaagggaagcaaccgggccggcaagtcggacgcggccaaggatgcaccgctgtcgccgcgccaggagcgtttcgtcgaggagtacctgaaggactcgaacgcgacccaggccgccatccgcgccggctatgccccggggaaggacaaccgggcagcgcatacgcaagcaaataggttgctgagttatgcgggggtaattcgcgccatcgaggagcgacgcgcaaagctcgggaagcggttcgagctgactcgcgagcggatgatggaggagtattgcaagatcgcttttgcggacccgcgaaacttctttcgcgaggatggcacgctgaagctggtgactgagttagacgacgcgactgccgccgcgctcgcgcacttcgaagtgctggaggagttcgacggctcgggccaggaccggacgcaggtcggctacacgaccaagatcaagtgggcggacaagaaggccgcgctcgacagcatcgcgcgcgtgatgggctggaaccaggacaaggtgaagctccagggcgatgcggagaacccgttgaccatgctgctgaagcaggtgagcggcacggcgttcacgccgcagggcgagggcgagccggaagactgagcctgatgacgatcgcatgaaccccgctccggcggggtttcgttcgtcataggcaaaagcaatcggggtggatagcgaaaatcaatcacgtgattcgttgcatgacctacaattcagtcaacgaacgaattgtcgttcgctcacgataggggatgatgatgaagatgcacccgaaaccgatggtccgccacgcgcaagtgcgcaagtgcgctgcgctggtagtgaccgagcgcggtaacgccgaattctgatcatgaagatcaccaccaaattctcccgcctagcggatttcaaggtcggcgtgcgggttgagtaccgcgacgcgcacgggcagctccagacgggcacgatcaccgacacatcggggcttgggtacgaggtcgagcgcaataatgccgatggctcgaccacgaagtgtcgcccgatcttcgcgttcgacatcacacgcatcatctgacaacagcccgccgcgtgctggcatcatcacatccagaccatgaccatgaccaccaaccaagccatcgagcggcaggccgagcaggccaacgccgccaccgcgctccagcaggcgctggcgaagcaaccgcagatcgagaccggccttgacctgctgggcgagggcttcctgggcgacgtagccgtcgagttctacggcaagctctacgacttcgggcgcgggccggccgtcgacatggaggacattgcgctcgccggcacgaccgtcagcatgcgcacgctggtgagcgtgaagcgctggacggagatcgagacgcagattctcgatcgcatcctgcgcaacctttaagcaacgtttaacagttcacgcccgccgcgcgcgggctttctcaaccgcagcacattcaggagtgatcatgggcatcatcaagctcgcgatcggcagcatgttcggctccaccgaacagcccagccagatcggcacgaccaagcgcaacggcggcagcgtggcccgcgacaagcgcgccgcccgcaaggcccgcaacgcgaagcgccacaagcgcgccggcaagtgagccgcgcgagcgacctgctgcgcgccgccaagcgcggggagatgcagaacgacagcgtttcactcgcgctcgacgttggcgcaatcgtctccttcggcatcgccatcggcaccgccatctacatctacttcacgctatgaaaccgttcgactccaaccaattccaggcaggcgccacggcgctcacgccgtccggccgcaaggtcacgttcgtcgccttcatcgccagcgtgcccgtcatgtatccgattgtcgcgcacatcgagggcgatgattggctcacgtcggtcagcatcgatggggtgttgcagaacaccatccagctcaccatggcgccggtcaaacgcacggtgttcgtgaacctgtaccgcgaagacgacggtcgcatcgtcgcgtcccacgagacgcacagcagcgaggatgaggccacatacgatcgcctgtcctccgactacatcggcgcattccccatcgagatcgaggaatgacaggattcggcatgcgcctgtggcgcagccggcatgggctgacccaggcacgtgcagcggagtgcctgggcctccaggccgtgacggtcagccagtacgagcgcggcgtgcgccgcgttccccacgtcgtcgcactgctgtgcggcgcctacaaccaccttgcgggcatgggcaactcgaccgagacgaccctgaccgcgctggagaatgcaagtgctgaaaaccattcttgagcagtaccgcaacggcgagcgcgggatgccgacgtatgacgagctggcggccattgcggatgagagcagcgccaagcgagccatcaacgccgcgatcgatgagctggcccgtgagcccggcgcggtcgggttcatgcacagcctgccgccgcaccatacgcgctacgtcgctggcggaagcctcggccagatcgtgagtatggcgaggcgccgaaaggacgagggactggcatcgattgccggcggcgatcacgatgggcatgcgcggatcaatggtggcgcctgccgcgcgcaggctgatgcgcaaccggctgggcggccagaagcagaggctctgcggttgactgtcgaagatgcaatgcgaatcgtgatggcgaactggggagataagtgcgccatcgaaaaagcgttcgccgcgaacctcccccgcaccgccgcagatgtgcggccaacagcgtgggtgcgcttctgcagtgacggcagttttgacggcccgatcatggataccgacatccgtatggccggcgctcgccgaacgtccggcgcatggacgccgctctatgtccatccctccgcagagatcgcccgagccgacgaactgcgtcacgccctgtccgtcgcacgcaaccacatgcgcgttatggcgaactgggtcaagttcagcgaccccgccgcgtacagctggtcctgcaacgccatcaccatcgtgaatgccgtgcttgaacgaggagagccgaaatgatcgagcatactggtcctgaaacgctcgtcggcccgttccccggcgctgtaccgcctgcgcggcccgttgggttcattgagcaatgggaactagaccagcttaaaaatgggtgcgtggcccgagtattcccgaccaaagatgagccacacgatgtggcgctattcacccaccccgaagcatcggcgacggggctgagtgatggctggaagcaagccgctgaatgggtgcgcgacaactatcaagactacgcaaacatcgcttccctggtcgatgcgatgctcgcccgcgccagcgccgcgactgcctgcgttgcctgtgaaggttcgccatcttctgaaaacaacccctgtgccgtgtgcggaagagccgcgactgtgggcgaggcgagcgtagccgacgatgacgtttttacatggctcgaaactgagatttctgctgtcgattgtcggtaccacggagaccctagctacgaccacgacgcctattggatgcgagaacgtgttttgaagctggtgaaggaagccaaggacgtatttggcaaagtcgcccagcagcaggccgagccggggcggatgagcagggtgacggctacgctggggcgcgcttgctcacaatttgccataagtttggcgtcactgttccagacggttggtcgtgggaaacgatagatgcagcgcgctttgcactgttggggcagattcgtagcgccattgaaaatagcaagccagccgggcatttcttcaaggcgtcgagcctgcatccgtgggtgcaagttgagaaggacaacccgaacgccgtgcctctgtatcgtgccgcccagtctggccagcagaaacgtggcgcgccgaagcaaatcctggccaagcgcaagctgacctgtgaggccatcgaaggcgcaattgctttcggctaccagaacaccaacctgccgccgtccgatgcccattggctggcaccgttctggaagatcggacgcaaacagtcggagcttgagcagtccggccagcgggcgggcgtggcaccggaatacgtaatggtgcagcgtcgtatgcgtccgacgcggcgacctgaaggtgaaggctggacggattgggaagagtgttcggaagacatcgcacgtgattgcgagcgagtgccagtatttcacggccagcaacacgaagtccgctggctgttcgccgcccagtcaggccagcgggcgggcgtggcggagggtggcagttggtgccgaagaaacccaccgagcagatgcagattgcggggcgttatgccactcacgacaatgcatgggagcaagcacgggcaatttgggctgctatgctcgccgccgcccccacaaaatgaaatgctggaaacacgcagcacccgcagccattggagagagcggacatgagtagaacgactgtgagcgaaatcctgtttggcgacaggaacaaggaagagcgtgcgctgcttctgcactggtgcgaactggccgcgtctcaaggtgggtcgttcagcatcaccgagcgctggacgggcaactggctccaggtattcacgatcaattggcccgatggggacaacgccgcccacggagagagccatgagtgagccgaagctgttgccgtgcccgtttgacggaggcgaagcacgtcggcctgaaggccggggtatcccgcgcaatctgatgatcgagcatactggtcctgaaacgctcgtcggcccgttccccggcgctgtaccgcctgcgcggcccggtgtgtaccgcgcccagactagatcgggcctcttcttttattcgtattggaacggatcgaggtggggcgcgccaaggctcacagcggacgacgcgtacgtagggcgcagcatcgcttcctgtcttcggtgtgcccggtggtggggcctggaggaagagcaatgaccgacgaccaaatccgcatgatcgcagagcgcatggacctggaggacagggtgtgccggctgcgggcgaccctgcgtgctgccgagcagatcggaccactgggcatcgaatacgccgaacgagttcgcatgcgcaatcagatcggcgccatggtgatgtacctggagattctgaccgatcgcatcaaggctttccaaggagaaccgcaatgacgcgcgaagaggcggtatcaatccgatacgcgacgttcgacgcgttcgcgatctggttccggttgtggtggggtcgcgagcctaccgcacgcgaggtgtggcgttatctcgccacgcggaccagcgaggctcccgcgcccaccccccagcagcaggccgagccgggggcggatgtgccgagcgacccaacaggcttgattgcgcgtctgcgcgtccatggggaagacaggaccaacactgcgttcgcaagatcaacaatgcgtgaggcggccggcgttttggaggaccagaagagagccggtccagttgggcatgaggcgtggccgaaagtgctgggcgtaggccgcgacaacgaacactcccgcaatttggtcgtgtatctcggcgcagcgcctaccgatggagaattgcgggcaatccatgacgcgcttcacgccgcccagtcaggccagcgggcggcggatgagaaggcgtcagagtgcggttgcgttaattcactgcttcagtgccacgaagcgcccggctgccagcttggtttagccgcccagtcgacgaagggacgcccgggcaacgcagggggcgagcaaccatgaccgcccaacaacaagacgacgaatacatccgtggctacaacgacgcatacgccgcgctaggtcgccgcgtcaaacccgagcagctgcgccaggagatcatgggccgcatcgccgcaggcacaggcccggacgccacggcgtacagcaacggcgcgatgaccgctgtgctcgaatggatcgagaggagcgtgcactgatcatgagcgacatctacacactgaccgccagcgtcaccagccgggccgtgatccgcaacaccggcctgggcagcatcgaggtgcgcgtccaccctgagtacgtcgagcgcatccgcgagctggtggccgagcacatgccggccgattcgcgcgtcgacgtcaagccgctgtcgcacgaggccatctacaccaaccgcatcacactgatcgacaatggggtccactgatgccgctcttcttctatcccatgttgttctggtacatcgtctgcgccgagttagccggcaagcgccgcaagcgcaaccgctcggaggaccgggagtgatggcgcaactcacgcaggcaatgtgcaacgcgctccgtcgcgcaccgcgtgattgggcgcgcttcccgccgtacatcgaggcgtcgccgcgcgtgatcaaggcgctcgccgcgcgcggcctcgtcgagatcaagcgcgtgacatggctcggcgagcatacggatatgtggcgccgcatgcgaaaagcataaagcttgcatggcgttgcaccgatccgcaccatacaatacatggtgttgacgattgcttttggaggcgcgatggagggcggcactagaatattcacattcccgaagaccgaggaggagattgtcgcctgcctgcgcgatccgcagtggcggatacggcacttgtacaagatcaagaacaagcagaagcaggtcgtgccgttcaagccgaacgacgcgcaggaggagcttctgcggcgcatgtggtttagaaatgtgattccaaaagctcgccaaagagggttttgtctgcatccgtctacacgcgtacttactgcttcccttgagtgggtgccgatcggagacataaagccgggtgcgcggctagtcgccgtcgatgaggacgtcccaggcggcaaaggcccgtcgcggaagatgaggacagcaacggtagaggccaccgcaatcacgcgtggtaacgcctaccgaatgctgttcgatgacgggcgtgaagttgtgtgcaccgggaatcaccgctggctcacgcggccatccgatgctgtagttggcaccaagtggcgctccatcgagtccgataacaagtgcaagatcaggccggggactcttgtccgttgggtcacaaagccatgggtgaatcctcatacgaggatgggtggatcggtggaatgctcgacggcgagggccacatgtccaagcgcaatgtcgcggcatcaatcaacgtcagtcaagttgtcgggccagcgctggcgcgcttcgagatgtacctcgacacgcgcggctacaactactgcacggaggacgacgctgcgacgcgagaaagcaaacaaggaagccagccggtcccgaagttggtggtcgggcgcatggatgagatgttccgcatcatcgggcagacgaggccgatcaggttcatcgggaacgaattttgggacgggcgagaccttcctgggaagcgaagcggtattggttgggcgaaagtcgcatcgatcgatccgcttggcgagatggatatggtggacttgcagacgtcggccaagaccttcatcgcggagggttcgtctctcacaactcaactctcattcagctcatgggcctggacaccgcactgttcaagccagggtcagacgtcggcatcatcgcccaggacttgccgaccgcgcaggagattttcgagagcaagatcaagctcgcatacgacaacctgccggacaccatcaagcagatggtgccaataacgcgcagcacgacgacggagatgaagtttgccaacggctcgggcgtgcgggtcggcacatcgatgcgcggcggtacgccgaactttgtccacgtctcggagttcggaaagatcagcgccaagtacccggacaaggcgcgcgaggtgctgacaggaacgctgcctgccgtgccgatcgacggcatcgtattcgtcgagtcgacggcagagggccgcgacggcgcgttctacgacatgtcgcacgatgccaaggccgcgcaggacgaggggcgcaagctgacgccgctggacttccgcctgcacttcgcaagctggtgggacgccgacgagtacgagctggacccggcgggcgtcatcatcaccgagaaggatcacgagtatttcgacaggatcgagtctctgatcgggcaggcgctgccgccgcgcaagcgcgcttggtacgtgacgacgcggcgccagctattcgccggcgatcatcagatgatgtttcaggagtttccgtcaacgttcgatgaggcgttcagcatcagcatggaaggcacctactacgcccagcagctcgcgcatgcgcgcaaggacggacgcatcatgcgcctgccggtgatgccgggcgtgccgtgctacaccgtgtgggacatcggcaacagcgacggcacggccatctgggtcgtgcagaagatcgggaatgagtggcgctgcctccgcttctacgaggcatggggcgagccgtacagccacgccgtccgctggctgcaaggtctcggcatggtgtgggacacgatgtacctgccacatgacgccgaccacatccgccagggccagaccgtcaacaagagtccgaagcagatgcttgaggagctgatgcccggcgtgcgcttcgagatcgtgccgcgcatcgaggacgtaaattggggcatccagcagacgcgcgacatgttcccgctgctgtggttcgacgatgagcacacaaagcccggtatcatccacatcgagaactaccgcaagaagtggaacgaccggcaggcatgctggtccaccgtaccagacaagaccggcggccactctgaggcggccgacgcgctgcgccagctggcccaggcgtatgccggcggccttatcaacgtgaatcgtggcgcacacaaacgcaagcgcccaagcagctggagaacctcgtgagcgaacgcaaacacttccttgacctgacccgttgccacttcacgcgcaacatcggcgacatcacgctctacggcacgtgggtgccgaggggtgacgacgacgaccacaccgagccggcgctggtgctcatgtcgcgccgcttcggcggccggccgtgctgcgtggcgctttcgagcgcatacctgtacaatgacccccgttattgtgcccgcgccgcacacgaattcgcgcagggcatggggttcgagccgggtttggcgctgacgcaccggattgcgaacatcatccacgaccatctgcaagacctgatcgagatgccgccggagccgacgcaggccgtcgtcgtggccgatgccacggtcgacatcgacggccgcaagcgatccgtggagatcatcagccacgagcaggccccgatctaatcgcctggagaacgcagtgttcgatttgaacgacgaccagaacacgcgggtgatcgcggaccatcccgaggcccggatgcccaccgaggagccgggcaaggatgacgcgccgccctcgcatcctctcgacagcgaggagatgaaggagctgcatcgtcgtctccggtcgtacctgcaccaggagctggaccgccaagccgagaaccggtttcagatggcggtcgacgaggactactacgacagcatccagtggacggaggccgacgcccaggtgctcaaggagcgcgggcaggcgccgctcgtcttcaacgtgatcgcgcagtcggtcaactggatcatcggcagcgagaagcgcgggcgcacagacttcaacatcctgccgcgcaagaaggccgacgccaagccggccgaggccaagaccaagctcctcaagtacctgtcggacgtcaaccgcctgccgttccaccgctcgcgctcgttcgaggatacggtcaaggtcggcctgggctggatcgaggacagctacgacgacagcacggacggcgagccgatctactcgcgctatgagagctggcgcaacgtgatcttcgacagcgcctccaccgagcttgacggcaccgacatgcgctacatcttccggccgaagtggctggacgttgacgtggcgtgcgcgctggtgcccgaccgcgccgacgagatcaagaaggcggccgtcgccgccgagcggtacggcaactattccgaggaagacggcgacgaggccatggattgggccgagttcgaccgcgacacctacagccagtcccgcaccgtctcgacccacaagcgccagcgcgtgcgcctgatcgagtgctggtatcgcaagccgatgcgcgccaccaagttcctgagtggcgacctgcgcggcgagacgctcgacgagagcaacccggcgcacgtcgaggcgcgggacagcggcctgtacagcctgggcgagcgcatcaccatgcgcgtgcgcgtggcgattttcacgagccgcgacctgctgttcgagggcgccagcccgttccgccacaaccgattcagcctcaccccgatttggggcttccgccgtggccgcgacaacctgccatacggcgtgatccgctggatgcgcgacatccaggacgacatcaacaagcgcgcgagcaaggcgctctacatcctgtcgtcaaacaaggtcgtgatggacgagggcgcggtcgaggacatcgaggagttccgcgaggaggtcgcacgcccggatgccgtgctcgtcaagaagccgggcaagcagatcgagctgaacgtcgatcgcgagctggctgccgctcacatggacatgatgagccgcgacatccagatgttgcagcaggtcggcggcgtgacggacgagctgatgggccgctcgaccaacgctgtgtccggcgtggcgatccaggcccgccaagagcagggcacggtcgccacgaacaaattgttcgacaacctgcgcttcgccgtgcagatgcagggcgagattcagctgtcgctcatcgagcagttcgtgacggaagaaaagaccttccgcatcaccaacgagcgcggcaaggccgacttcatcacggtcaacgacggcctgccggaaaacgacatcgtgcgcacgaaggccgacttcatcatcggcgagtcggattggcgcgcgacctaccgtcaggcggcctccgagcagctctcgcagatgatcatgaagatgccgccgcaggtgggcctcgtgatgctcgacctgtgggccgattccacggacctgccgaaccgcgacgagatcgtgaagcgcatccgccagatcaacggcatgcgcgacccggacgccacggagccgacgcccgaggagctgcaacagcagcaggccgccgccgagcaggcgcaggcgcagaaggcgatgttcatggccgagctgtccgagaagcagggcaaggcggacaaggcccaggccgatgccgtcgccgcgcaagcgaatgcagacctgcgtgccgcccaggcggaacaggtgcgccgccagacggtcaacaccaacgtcacgtcgatcgcggcggccatggaggcggccaaggccatcgtgaccatgccgaccatcgccaaggtcggcgacgcggtgctggtcgaggccggctacgagaacaacggcattgcgccggcgggtggactgcacacgccagcgccgcagcaggcggtgaacccggcagctcaagggcttccgccacaacagccgcaacctcaacagccccaggagccggccgtgtcgccgtctcccgagcagttgaatggtgcggcaccgagcaataccggcgaacccgtgacgccggatcaaactctccaacagtgaagggactgagatgacgaccacacacagcgaagaactgctttccggcctgacgcccgaggagctggccgcgctcaatgaggacgacggcggcgaaggcggtgacgcgggcggcggcgatggcggccagggcggcgacgatggtgcaggcgctggcgcagatgatggtgctggtgccaaaaacgccggcggcgagggtgaaaacggcgccaatgctggcgatgatgctggtgccggcgccggtgaaggcgctggtgctggcggcgaaggcgcgggcggcgaaggcgatgacggcgcggtggacaccggcacgaagtcgatgcccgtgtacgtcgccgaggcgccggaagacgccgacaccaagctgaagggctggccgacaagcgggccgagctgcgcaagcagtacgacgacggcgacatcacgttcgacgaactcgaatccgcgcgtgacgagatcacgaaggaagagaaggcgatcgagcgcgcccagctcaaggccgagatggcggccgagatgcagcaacagcagacggtcaacgactggaaccgagacgtcaactccttcctggacgccaacccggaataccgcgccagcgacgtgcgttatcaggcgctcgacatgatggtgcgcaagatcggcggcgctcctgaagcggcgaagatgaccggcccgcaggtgctcgccaaggcgcacgaggagctgcaaaaggcgttcggcgtggccgcgccggcgaagaatgacgagggcaacggcaagggcgagaagaagccgcccccgaaggtcgacgttccgccgacgctgggcaagctgccggccgccgagaacaacgacacccagggcggcaagtacgccgcgctcgaccgactgctggaaaccgatccgctcgctcacgaggacgcgctgatgaagctctccgcgtccgagcgcgagtcgtatctggcatcgcgataaggaggagtgatgcaaggcgcatacatcgcaaacccggtgcgcgtcagggcggcgcgcatcgtcgaggtcaacggcgcgctgttgacgctggagggtggcggcatcttccgggcggacgagggcatgaccgcgcgctacacgccggtgccgggcgactacctcgtgacgcaggaggacggctacgtctacgtgaacccgaaggacgtgttcgagcgcaagtaccgtcccgaggcggtcgaggcgaaggccgagcttgcgccgcaccagcagcgcgtcgtcgacgagaaggccgagcttgacgatcgcattgccaagctcgccgccttcatcggcaccttcatggagccgctcacggtgtttggcagcctgcccgagcccgagcgcatgcgcctgtacgcccagcaccgcgccatgacgacttactcgtccatcctgggcgagcgcatcgccgcattctgacgggggaatatgggcacgctccgcatcgacctgaagccaggggagagcatcgctatcggcgacttcgccgtggtgacgcttgagcacaagtcaggccagatcgcccggctcgcgtttcaggccgacaagtctgtgcccattcggcgcgcgacggcggccagcccggccaagatcgcggcctcggtcggcatcaccgggaaagcgtgacgatgccccactacggtggggtgtcgtgaccgcattgcgcgggcgacaattggtctccattcacaggagggccaaatgaacgagcaatgcaaatacatcgacgaacccgccggataccagccgaccgagatcgacaaggagtttttccgcctggacagcacgatccagatgctgaaggacgagaccaatgcactgtccgatcgactggtaagcgtcgtcgttccggaatgtgaaagcaccaacgaaagcaagattgactcttcgtgcaacacgggcctcggcctacgcatccgggcgctggacaacgaggcgcacaaggcgctcaaggtcgtccgcagcttgctggagcgcatccagctctgagcccgccggcgccgccatagccccgcccagcgctgggttttcttttcccggcgctgtgttgcaaatcagaacgcgcggtaataaaatcggaccagtcgaccaacgcaggagtgtgggttgatccctatcaaccattaggagcacactcccatgtccagcaccgttatcgcgttcggcgatcccaaggcccaaaagaagtggtcttcggaactcgccgtcgacattcggaaaaagagctacttcgagtcgcgattcatcggcaccagcgaaaacgccgtcatccagcgcaagaccgaagttgagagcgacgccggcgaccgcgtgtcgttcgacctgtctgtccgtctgcgcggccagccgacgttcggtgacgatcgcgtcgagggcaaggaagaaaacctcaagttctacaccgacgaagtgatcatcgatcaggtgcgtcactcggtgtccgctggtggccgcatgtcgcgcaagcgcactgcgcacgacctgcgcaagaccggccgcgaccgcctgggtgactacttctaccagctcaccgacgaactgttcttcatgtacctgtcgggcgcgcgcggcatcaacaaggacttcatcctgccgacgtcgttcaccggctacgcgaagaacccgttcaacacgccggatgcagctcacctgctgtacggcggcgtggcgacgagcaaggccagcctcgccaacaccgacaccatgtcgcgcgtggtgatcgagcgcgccaacgtgcaggccaccatgatgcaggcccaagacccggaaacggcgaacatggtgccggtcagcgtggaaggcgaagaccgctacgtgtgtgtgatgtcgcccttccaggagcacagcctgcgcacgtcggacgccgccggctggctggaaatccagaaggccgccgccgctgctgaaggccgcaacaacccgatcttcaagggtggcttgggcatgatcggcaacacggtgctgcactcgcaccgcaacgtggttcgcttcagcgactacggcgcgggctctgaccagccggctgcgcgtgcgctgttcatgggtcgtcaggctgctgtcgtggcctacggcaccaagggcggcctgcgctacgactggcaggaagagacgaaggactacggcaacgagccgaccgtcgcctcgggcttcatcgctggcatcaagaagacgcgcttcaacgaccgcgacttcggtgtgatctcgatcgacacctacgccaaagacccgaacccgaacaacccggcttaaccggatcggacgcaaaggctggcggcttcggccgctggctgtagcaccgtgcaatccaccttctcaggagcaacatcatggctttgatccaatccgatttcgcgcagggcattcgcatgacgcccgtgcccgattgcgctggcgacgttaccgcctgccgcttcgacatcacgctgaagaacgcgccggccgctggcgacatcatcgagcttggcgttctgccgggcaacgctgtcccggtggaggcgatcctggacgtcgacgatctcgacaccggcggcgcgccgaccatcacgctcgacgtcggcatcatgtccggcccggtcggcaagaacgacccggcgcgcacctgcggcaacgagctgtttgccgcgtcgactgtcggccaagccggcggcgtcgtgcgcgcaacggcatcgagcgccttccgcatccagaaggcggaagatcaccgctcggtgggcgtgaaggtcgccgccggcccggccacgggcgccgctggcaagacgatcgcgctgatcctgttctacgtgcagggcaccagccagtaagcgacgggcgcaacggcgggggcttcggcccctgcttttccaactgagggatgatcatgcagatcgaatgcattctgaagcgccaaggcggaacgaaagtgacgctcgaaggcgtcgaataccacttcgcgccgctggaggatgccgcgcacgtcgctgaagtcgagagcgagccgcactgcaagcgcctcctgtcgatcccggaggcgtaccagccgtatcgccctgcctcgtcgacaggcgcgccgcagacgctcgtgccggccaacaagctgaccaacgccgagacgctgctgggcagcgacgtccacccggcagtgatcgacctgggcgacagccgcaccgtgcagctcggcgatgtcgttgcccgtgcgcattccgcgtccggcctgaccgtcgaagagtggaacgcgctcgacgcggatgcccgccactcgctgatcgacgccatgctcgacacgatggcagacgagatgccgcaggcgccggaccgcgccgcgctggccgagcagtacaaggccaagttcggcaaggctccgcacggcaagtggaccgtcgaaaccatccagcagaagctcgcttcgggcgaggcataagctatggcattccccgccagcgtcgtgctctcccgcgccgcgaccttgttgcaagacgaggatcacgagcgctggacggtggatgagctgcttgagtggctcacggatggcacgcgcgagattgtcgtgcgcaagccgtccgcgtacatgaagacgacgaccgcagcactggccgccggctcgaaacaggcgttgcccgaggacgcgatccagctgatcgatgtcccgcgcaacctgaaggcggacggatcgccgggccgcgctgtcacggccaccgatcgccgcctgctggataccgagaacccggactggcacagcatgaagccggccgggcaaatccgacactacacctacgacagcaacgttccgaacgtcttctacacctatccgccggccgccgccggcgtgcaggttgagctggtctgcgcgtggcggcaccccgcgctgacgacgcagaatgacgtggtgcagatgggcgctgagttcgtcagtgcgctggtgagctggtgcctgtaccgcgcatcgagcaaggacagcgagtttgcgaacggtgccgtggccgccgcgcactacagcgcgttcgctgacgcgctcggcatgcaggccactggcacgccgacaacccaggccgccgccgctgctgccgccgccggagccgcgcaatgaagccgctcgacgtgttcatgccgatcatccaccggtttgcgcccggctgcccggagcctaccgcgttcgctgcgatccgcgaggcggccatcaagttctgcgagcgcacgcgcctgtggcgctgcgacgatgaattcgacgtgaccgccaacgagtgcgcggatgtcgccgtgccctatggcgctgcgctctacgagatggagctggtgcagttcaacggccgcgaccttcgccccgtgtcgacgcaatggctcgacgagcaggcgcctgaatggcggacaaacacgcaggccagtcaggcgcagtacgtcacgcagtacagcgaggacacgctgactttctacccgcctgacaacgggcgcgtgaaggtctacggcctgctgaagccgacgttggatgccgacagcctgccggatttcatcgccgacaagtaccgcaagacgatcgccgatggcgcgctctccgagctgctgatcatccccggcaagacctggatgagcgctgaccttgctgtgttcttcggcacccggttcgaccgcgagctggaccgcctttctaccaagaccatcaagggccagcagcgcgcacctgtgcgcacgaaggcacaatttttctaaggagctgtcatggccgccgcatccacctacaccgagaacaacatcctcaatgcgctgctgcgtggcgttgcgttcccgctgccagccaagacctacgtgtcgctgcacactggcgacccaggcgtcggggccggcgccaacgaagtcagtctgagcaactggccggcctacgtgcgccgcgaagccgagcagggcggcgcgatcggctcgggctggacgccggcggccagcggccagacgagcaacgtcaaccagctcacgtatcccgctaacaacggcgtggccgcagtgaccgtgacgcactatgccgtgttcgacgcttccaccggcggcaacctgctgttcaaggcgccgctgaccgtcgcgcgcacgctccaggtcggcgacgtgttcgtgttcgacgtcggtagcctgacggcccaggcgtcgtaacgcgcaatgcgctacgccctcaatgtcgcaccgatcaatggctgggaaacgcatctcggccaaggtgtagctgcgctcgcggccgacgccacgggtgtggcgacgccggcagcgcaggggggcggcgtagcgcctatcatgctcgatggtgccgcactcggaaacatgcggcaggtgctctacggggccgcgcagcttggtgtcgatgcatacggcttcggtcgcattgcaggccccggcggcctgggcctgatcggcctggatgccatgggcgcggcgaagctgatcgcaaagccaacggcatcgtctcagatcgacatcggggcggctggcgatagcaagattgccatgctgctgagtggagcgggacgcatcgacctgactggctcgggtctcttggaggctgccatgttgggttctggcatcggaagtatcgaactgtctggctcaggcgacgcgcgcacgcatcacccggtctacgggggcggacgcgccgacatcctgagcaacgcgaccggccgcgccgtcacgatcggcaagaacagcggcgccgcgagcatcgccatggaagcgcaggggctaggccgcctgggcgcgcacgcctacggcgctggtgccgcgcaggtcgtcatggacggccgtggcgtgccgcgcagctaccgcgtcatctacgccggcggcagcgccgtcatgtcgctttccgccgcgcaccagatcactgccaccatctccggaccgttcgagccggcgcctatcgagcgtcagctgttcgtggaggatgacagccggacactcgtggtgtcgagcgatggcgaccgcgtgccgggcggccaagtgctgccgcgctactcggatggcaccatcgcgctcgatctcattcttgacgctggccggtatggcgaggccagcaccaccggcgcgatcgatcgactggatcagatcgttggcgccgacatgccgaacaattcctgggggaacccgtgaccgaatcactgaacagccgcgtcgccaagttcgacgagatcgtcaccaacgttgacacgtgggtgaaaggcgggagcgccgacaccgttgatttcggtggcggcccggtcgattcgcccgcaaagctcatcgagggccagcgaacagacatcgctgctgctgctgcggcgctgatctcgcagaaggacgagcagatcaaccagcaggcgaacggcgtgctggcgcaggcgaacgcggcggccagcaatgcaaacggcagcgctggcgcggcgtcggcgagcgcagcgagtgcggcagcgtcggcggccaacagcgctgcgagcgcggcagatgccgccgcatcgtcggctcagaagttcgtgacgtcggctggcgcggcgaaccagaagccgaacacgtcctacacctacatcaacccgctgtcgcgcgtgtgcaccatcaccggcggcggagacggctcgaacttccagatgatcgggtacggccacaaccgccagaagtttctgggcgcggccaccactggcgtctggaactacctgccggtcaacggcatcgccaacctgaatttctacaggatcgcgggagatggcactcgcacaaagctgacgtacacatccggcactccgaccggtaatcagtggtccggcaccgtgggcgccagcagcgtcacctggacgctcggcacggccctggacacgttgagcaaactgttcgttgaagacccatttgccacgatcccgacgacgggcacgcagcctgactactcctggatcggcggcggctatgacaatgtggtcgataggggcgtgatgcagcatgtcacgggcgcgcacaatcgcgtgctcgacggcgaccacaccacgatgctgggcggcagctaccacaccaacgacgggggcagctacaacgcattctgcggcggcaccgacaactggaacggatcgggcgccacgctgggcagcggtcacttcggcgggctgcgcaactattgcaacggcgccacctctggcacgacgttcgcgcagtcctgcaaggtgaactctgcgtacagctggacgcacggctacgggaatactgccaatggcaccgccgtctccatcggcgggcgaaacaacaccgcatccgggtcggactcggccgtcttcggcaacggcaacaccagcaccgcgaactactcgttcgccatcggcaacggcaacacggtcggccatcactacgcccgcagtctgggcgggtacaagggcagcacgctgtatcagggtgagctgctggtctgggcgtaccgcgacacgtcggtcggcacgaacccgttcgtgagccgccggcaggtcccgctgtccgtcgtcaccagcaccaccgcatcggcactgctagccacgctggacggcatcaccaacctcacgatgcccgtcagcaccgcctggaagtgcacgctggaggtgttcgcgcgcagcacggccctgcggtgcgccgcgttcgagctgaagtttcgggccagcaccgattcatcaggcactctgatcctgggcacgccgaccgtggtctggtccgacgtggacgcgaatttcggcacggctgccaacgctgcctcggtctcggtagtcgccaacggcaatgccatcaactgcgtggtgcagccacgggtcgattctccagtcggcgcaaaatggggcgccgtgctgcacatcaatgaggtgaactgatgctgggtatcgttcagaaaacggccacggaacagctcgactacgacatcgatttcgcgcgctggatgcctgacggcgatgtgctccagtcggctggcgtcgttatcacgccggacgacggcacgcttacctcgccggcctacgatatcgacgggactgtcgtgaaggtatggctcgccggcggcacggctggagccagctacaatgtcgacgtgacagtcgcaacggccgccggccgcatcaaggaaacctgtttcaagactcgcgtgaggagctgctgatggctgccgttcaattcgcaaacaacgctgctagccgtcttgtcggcccgctggcgccttccggtacgtcattgacggtcacgcctggggatggagcgcttttcccgacgctaggcgctggcgattggttcatggcaacgctcatccgatcggatggcgcgcgcgagatcgtcaaggtgacgtcgcgcacggtggacgccatgtccatcacgcgcgcgcaggagggctcgtctgcgctgtcgttcaacccgaacgatcgcatcgaggcgcgcctgaccgctggcgaactcggcgacttccgtgacggcatcgcgtccgcacagagtgccgccagtgccgcgcagggcacggccgatggcaagatttccaaggctggcgacaccatgaccggcaacctcaacatggagggcgccgcgccgattatcacgttccgcgagacggatcaggccgctccggcggggcgctggcgcctcgtggcagatggcggaaactggtcgttgcgccggagcacggcgggcgagttcgcctccgagaacagcacgatgtggttcggtccggacgacagcgtgcacttcttgaacgacattctgatcggccgtggcaacctgggatcggtgtatgacgcgctggcgagcaaggccaccagcgcggctctggcgagcggactggccgccaagcctgatgcggatggtgtttccgttactggtttcgtcaacggcaatttctacgagccgtatttccgcaagtcctctgacaacaccgttcggcgtctcgtggcgaacaccagtgcaaatgggatcgccctgtcgtggtctggatcgtttctgagccgaacgatcgacaacacggccactgcgacgatctgggacaccgcaaatgcgcccggcgctggcaccaccaacctcgacaagacattctactgcggcgacggcacgcgcatcgggcgcagctgggtgccgggcagcggcttcctttcgatcacggtcgacggcaccaactacggcatctcgatcagcgcttccgacgagcgtctgaagcgagagatcgccccgagcgaggcgagcgcgctgtcgaagctaggccgcattgagctgttcagcttccggtacaaggaaggcaatgctttcctggacccgtcgcagcaccacgatatcggcttcatcgcgcagcaactcgccagcgttgaccctacgttcgtggccggcggcggcgaaacgatgctgtccccgaaccttcagcccatcgtggcgacgctggtgaaggcggtgcaggagctgcggtcacaggtggacgccctcaaggcccaggtgggcgcatgatgaccatcccctcgcacttcgctcgatcacgcaaggagcctccgaccaaaggcttctgtgtgatttcgactattgcgaggggattatggctatcaagctgtccaacaatgcagtgtcccatcttgccgccgagctggcgcagggcgacacccaaatcatcctgacgccgggcgagggcagcatgttcccgacactcggcgccggagactacttccaggccacgctcgcgaaagcattcggccaatacgagatcgtgcgcgccactgcgcgcgacatcgacacgctgaccgtgctgcgagggcaggagaacacccaggcgctgcacttcgcagccggcgatcgtatcgagctgcgcgtcacggcgggggtgttgaatgcgctcgccgacgcggttcgccagattcgtcccaagccgggagacatcaagatgtggagcggcgcggtcgccaacatcgctgccgtgcatgggtctggatggcagcttgccgatgggacgaatgggaccattgacctacgggaccggttcatcgtcggggcgggtgcatcctatgcgcctgggaacaccggaggagccaacaccgtcgcgatttccgcagagcagatgccgcagcataatcacggggtcaatgaccccggccacaatcacggcgtcaacgatccgacgcacgcgcatagcgtctacgaccctggacactcgcacggccacaacacggcggcgctgactccatcgagcacgggcggcggcgcgttccagatcaacggctatgccggcggcacgatcaacgcttccgccacggggatcggcatctacggggctgctacgggcatcagcattcagggcgcggggactggcatcagcacgcaaaattccggcagcggcgccgcacacgagaaccgaccgccgtatttcgcgctggcgttcatccagtacactggcgctggtgtgattgaaatctagggcaacatgacatgacgacgctgaagttgaccgcttactctggtgaggtgccgcgcacgctgccacggctcttggccgacaccgcgtcgcaacgcgcgctcaacgtgcgcctggacaacggcggcctcacgccgacgcgccagcctcgattcgaggccaacatctccgtcgacaacgccaagaccatctacaagcacaacggcgcgtggctggcgtggcagaacgtggtgcacgccgcgcccggcccggtggcccaggaccgcctctactacatgggcgacggcaagccgaagatgatcgtcgacggcaccacctatgacttggctgtgccgatgccgactgccgcgcccgcgctcaccgtgaccggcacgggcactggcaacgtcaccagcattgcctacgtctacacgttcgtcaccgcgttcggcgaggaatctgagccgtccgcgctctccaacgtcgcgggctggcagagcggccagacgcgcacgctgacgggcatccaggcgccgccggcgggccgcaacatcaccaagcagcgcttctaccggtcgcagaccggcagcggtggcaccgatctgttcttcatcgaggagcgcgccgcgtcggcggccaatttcgtcgacacgcacgcgaccaacgacttcggcgagatgctgccgtcgcttgaatacaacgctccgccggacggcctgaaagggctgatctcgctgcctaacggcatgatggccgcattcacaggcaaagacctgtatttctgcgagccgttcatcccgcacgcgtggcccgagaagtacatcctgacgatggattaccagatcgtggcgctcggcgcctacggcaccacgattgtggtcatgaccgagggtctgccgtacatcgtgtcgggcaccgcgccggagaacatgcagcagcagcgtgtcgagctgaacctgccgtgcatcaacgcgcgcggcgtgatcgacctgggctactcggtggcatatccgtcgcatgacgggctggtgatggctggctccaacggaatgcaggtgatcaccgaacagctgatgacgcgcaacgactggatgaagaccgggccgggcaacatcgtcggcggccagttcaatggccggtatttcgcgagctacgagtacatcgagccgagcggcgccgctttcagcgggacactgattttcgacactaccggcgcagcgcccttcatcatccgctcgaaccacaaggcggacgccttctttcacgagttgcaaaccggcgcgctctacttcctggtcggcaaggagattttcgagtgggacgcgctcgggcaggtcaacgagacgctgagctggcggtccaagcaattcgtgctcccgacgccgaccaacttcggtgtcatcctgatcgagggcagcactgccgccagcgaagaggagcaggcagcatacgaggctgagcgccagcgcatcgagacggagaacgcgacgaacttcgcgctgccgtccatcggcggcgaactgaatggcgctgaggtcaatctgttcgcggtcaatggcgacatgatgcagcggctgccggatgaggggttcgtgagcgtgtcgatctatgctgacggcaagctggtcaagaccgtgagcaagatgaacaggatggcgcggctgccgtcgggcttccttgcgcgcatctgggaaatcgaggtcaacagcaacatcaacatctccgacatcgtgctggcgaccaccggccaagaactgaggaacgtgtgatgggacgcggaacggacaacatgatcgcgcgcaacacgtccatgcgctcgggcctcgacccggcgcaggccatgcgcaagtacgaccgcgccgtcgagcaggtacagattctcgtcggcgagcgcggcccgagcgacggatcgcagatggccgtgctgcgtggcgacgtgatgggtggcggccagcctgccgatctccagtcgacgcagatcacggccgcgccgaccgccgccgactacaacaaattgcaggcggatatcgcagcgatccattcgcttctgtctgcgatttacaacaccgtttctgcgaagaaacgttgacagcgccgcaaatccgcgattggcggagcggtacgaaaaccatacaatggtcgggtaattcgaccgttgcgagctatgaccacaaacatcgttttcgacgaggactaccggctgatcgggtgggcggccgagcgcatcggcatcgaatgcttctcccatgatgcgcacgcgatcggcctggagcgcgacggcgagctggtggccgtcgtcgtctatgaccgcttctcgccgcacgactgccacatgcacgtggcgagcgacggcaccaagcgatggctcgtgcgcgagttcctcgtcgcgtgcttcgcgtacccgttcatccagctcggcttgcgccgcgtcaccggcctcgtgccggcgagcaacgccgccgcgctgaagttcgacaagaacctcggcttcctggtcgagggctatcaccatcaggcgctcggcgacgacgacctgatcacactgggcatgctcagggaaaactgtcgctggattcctagcgaacatcgggagaatacgcatgggcggtaaaggcggcggcgacgcaccggcacccgatccgaatgtcggcgtagcggcgttgaagaacgcggagctgggccagaactggctcgacttcgccaaggagcaattccaggtcgggaacgtccgtcagtcggacatggatgcgctgacgaagcgcgtcaccgagcagcagctggcgacgcaggatcagtcgaaccagtgggcgcaggaggatcgcgcccgctacaagaacacgttccagccgctccaggacgagttcatcaagacggcgaacgagtacgacacgccggagaaggaggcgcaggcagccgccgaggccaagagcgacgtccagcgcaatgcaacgcagcagcagggcgccgaacagcgcaacatggctgcgatgggcatcaacccgatgtccggccgcttccaggggcagagccgcgccacgtcgacgctgaccgcactggccggagccggcgcggagaacgccgcgcgcgagaacgtgcgaaataaggcgctggcgctgaagtccgacgccatcaacatgggcaatggcctgcctgcttccgccgcaagcgcatacggcctgggcctgaacgccggtaactcggcggtcggcaacacgagttctgccaactccaacttctaccagaacaatgggatcatgagtcagggctatggtggagccatgcagggctacaacaacatgggtagcctgctgaaccagcagtacaacggtcaggtcaatgcttggtcggctcaacagcaagcgggcgcgacaagcggcgcgggcatcggctcgatggtcggcacgattggcggtgctgcgctcatggtgttctaaaagggaaaacatgcagaagatcatcagcaatcacaagaaaatcgcgctccagctctccggcggcaaggattcgctcgcgtgcctgtacatcatgcgcccgtattgggatcgcctgacggtgtactggctcaacaccggcgcggccttcccggagacggtcgagcagatgaaggcgatccgcgagatggtgccgcacttcgtggagatcggcggtcgccaaccggaggtgatcgaggaattcggcctgccgtccgacatcgtgccggtcaacagcacgccggtcggcgtgacggcggcgggcgctggccgggtgctgatccaggatcgctactcctgctgcctgcgctcgctgatgatcccgatgcacgagcgcatgatgcaggatggtgtcacgctgatcattcgcggccagaaggacagcgaccgcctgaaggcaccgatcaattcgggcgatgtggtcgatggcattcaatacctgctgccgatcgagacttgggatgacgatgatgtgctgtcgtacctgaatgggcagggcgcgacgctgccgcgcttctatgagacgctgcgcgcatcgcccgactgcatctcgtgctcggcgtattgggaggaggggcgcgcggcctacctgaagcaacaccacccgcgtgaacacgtcatctatctggcacggctcgacgccatcaacgtggcggtcaatgagcacatcgcggcattcaatcacgaggtgaacgcatgagcttttggggcggattcgccggcggcatgtcccagggcctccggcaaggcatggcgatgggcaaggacttgcgcacgcgcatgaaggaggacgagctggagcaggtgcgccagcagggcctcaaggaggcgcaggaggcgcgcaatgcggccatcgccgacatcgtgaagcagaacggcatcagcggcaccacgcaggcggcgcccgccgcgccgtcgcaggatggcggcaacgcgccagctccagctcctgacgctgcgcccgcgcccgcgccggtcgagattgcgccgtcgcccgcgcccgcgccggctccggcgagcagcaccgcgccgtccgcgaacatcgcgccggcgtcgccgtctgccgcctttggcctgatgggcactccgactacggaccgctcccaggagctgccgtccttcagcgttggcggcaagacgttcgcgaaccgcgctgatgccgaggccgaggcaggcaaacaggtgccgaacgtggccgactacttcatgaagactggcgtgccgaagctgcaagaggcgtacatcgcgcagggcgatttcgacaaggcggaatcgctcggcaagtgggtggagtcgcggcgcgggcaggatgccgtgaagacgttcggcaaggccatgaccaagctgatgttcacgaacgacgtggacggcggcgtgaaggcgctgggcgactactacaacaagttcatcgacgatggcgtcgacttcgtgtcgcacggtgtaggcgaggatggcaagatcaacgtcacgctgaagaacaagggcaacggcacggaatcgaacatcagcctgtccaagggcgatatcctgcgcatgggcatggcctacgatccggccaagctgcacgaaatggcgctgtcgcaggaggcgcagaacgtcaagaacgcggccgacgtcgccaaggaggaccggaagttcaagcgggacgtctccatgcaggtgctcaagggcaaccagcagagcaagctcgaagatcagaagtcgggcaaccgcatcagcgaggaaacggccaaggcgcaaaccgacgtgtcgacgaccggcgccaaggagcgggccaaggtcgaggcgcaggtaggcgcaaaggtcgacgctctgcgcgccgccggcgtgtccgacgacttcatcaaggaggcgctgcccggcatcctgggcgcgggccagtacaagcgcgccacgtcgccggaggaggcgcgccgcctcgcgcactccgacctgatgaagaacatgccgggctatgcgaacaagagcgcagaggatcagcagaagattctcgacaagacgatgagcatcatctcggctggcgggaagccgagccagactggtgtgcccgatggcgctaccgcgccggcgaaggatgccgcgccgccggcggcatcgccgaagggcgtctacgtgcgcgacaagaagacgggcgagatcaagctgatcgatccgtcgcaactcggcccaggccctagcccggcgccgaagcaatcggtgtacgcgctgcccccgcgcgacaagtaaaacaggcccgttcatcgggtacaatctcgggacactggacacagatagaggcgcttcgtggcagacaaccaaccgttcttcgctcccctgttcgacaccaccgaagacacgaacgcctctatctaccaactgcccccgctgcaagaagcgctgaagtccgagccggccgccgcgccggccgtgaagtcgcgtggcatgccgagcgccgccgacgtatcccgccgcgccgaggagctgggcatcgaccccaagttcgcgctctccatcttcaagcaggagtcgagcggcaacttcaacgaacgcgacagcaacaagggcgcgatcggcggcatgcaggttatgcccggcacgtacaaggccatgatgggcaccacggaaggcatgcgcgatccgtggaacaacctggaggccggcctgcgctacatcgcctacgggcagcgcacgctcggcacgaaagaccccgcgctgctggctgccggctaccacgccggctatgaccgcaaagacctgaagcagggcatcatcccggatgtgtcggatggcgcgaagcgcactcgagactacgcgcgcgaggttgccgcacgcgctggcggctccgctccggcgggcaaggtggcatctgccgacagcgcagcgccggcgccgccttccatcccatcgctgcaatccgcactcgatgcgcaggagccgggccgctacgaggtgctggccgatccgggcgccgacgtgctcacgcgtctcaacaccggcgacgacaaggaccgctacgagctggcatcgcccgacgagatcagccgcttcgaggccggcaagaaggagagcgtcggcgacgcggcgaagtcgttcctcggcaagtcgctcaaagcaggcatgtacgatctcgccggcgccggcgccaagctgctggatgccgtcaacccctggacgttgagcgagagcgacgcggccacgctgttcaagaacgatccggcgaagctgaaggactaccaggacaacagcgtcgccatgatcctgtcgcgcttcgcgcggcgcatgtcgcacaacagcgaagatgcgatgaaggagatttcgcccggcgccgagctgcgctacgggcacaaggaatacgccaccaccgaccttgacaaggcggcctatgcctcgccggtcaaggtgatcggcgacgtcgtgcgcaacctgccgacgcaggtgggcctcgccatctccgcgtacctcacgcgcggcgccgccgcccaggctgaagcccaggctctcaaggccggtctgacggccgaggccgcgaagcaggctgccattcaggcgggcgcgaagacgatggcgaccatgggcgcggtcagcgagggcgcgatcggctatgcgcagcaggcgaaccaagcgcgcgaggaggccgagaaggtcaagcaagccgacctggagaagtcgcccgagtaccagaagcttctctccaacggctacacgccggagacggccaagcagaagctggccgcagtcacggccgagcaggctggcgttgttgccggcgtcgtcgacgcggctgtgaacaaggtgggcggccacttcctcggcaagatcatgaccgagggcgggaagttcgtgccgcgcgtcatgaagggcttcgtcaatgagggcgccaccgaggccatccaaagccctggcgagcagtatgcgcagaacgccgccgtgaagcgcaacatcaacccgaaccaagacctgagcgagggggtgctggagaacgccgtgcagggcctagtggtcggcggcgtgtcgggcggcggcttcgctggcgccatggggcgcggccatagcgcgaacagtgccgcaccaaacaatgctgcaccggcagtgcctgccgcgcccgccgccgcagctcctgtcgcgccggtggccgagcctgccgcgcccgctggcccgatcgagcgcgccgtaggcaaggtcatgcccgagagcaagcaggtcgttggcgacgacggcactgtctacaacgtcaaggtcggcgacagcggtgttgaggtcgcggcgatcgatccggccgcccaggcggcgccggcgctgaccgagcgcgagaaggcgctgcaagagatcggcgacaaggctgccagcgagaagccgcccaagcccgagccggcgcccaagaaggaacaggagccggcggccaaggtcgaggaggccaagtccgcaagcgatgaggagccggccgcgaaggcgcccgccggcccgaccgacacccgcgcgcccgccggcgccgacctgtccaccatggacgagccggcgctgcgcgagcgcatgaagtatctcgccggtcaggcgaagcgcacgggcggctgggacaagatgctggtgaccgagcgcaagcgcgtcgaggccgagatcggcaagcgcaagaacggccagcccgccgcccaggtggagcaggccgccgagccggcgccggaaaagaccgcatccggcgcattcgtgaagcagtcggacgcaaacgacgcgatgcgcaccgccgcgcgcgataccggcaagccgcatcaggttgtcgagcgggaggccggccgcgccgtcttcgacgtgaggcccatggaggaagccaatggagcgaacgctcgccgcgctgaacctgctggagcgcctgtcaccgcagcagagcaggcaagcgcagctcctgcggcgcctgctgctgaacggccggcgcgtgccgcgaagactgcgccaactgctggagaacctggagcggctgctggagaggccaccgctgagccggctgcactgacagtcaaggcggccagcgggaaggtctatccgaccaagggcgagcggctgccgaaggccggggtcgttcctgcaaacctcggcaacgatggcatcgtgtacgtcggccgtccgaacagcctgcatttccatgtggctgacaggcactccaaggcagccggcggattctccgacacgggattcgtcaccccggatggtcagtacctcacgcgcgaggaggccctggcctgggtcaacactcatgaggccaaggtcaagccgtccgagaacatgggcgcgagcctcgacgcgatcgactaccgcgagcaggtcccggaatcgaagcgcaaggctccggaggcggcggaagacgcagccgccaagccggccgccgcgccagccgaggccgtgaagtggttcggcacgcgcgagaaggccgaggcccacatcgccaagaagggtatcgccgacacgcacgaggtcgtgcaggcgcagaaccgccgcttcgagattcatccgaaggtggccgacggcttcgagcggttcgcgcccgagaccggcacgctcggcatcccgcgcgcggagatgccgcaggtgccgtcgcaatcgcacggcgcgctggtcaaccacctgaacgcgcagggcatcgagcacgagaccaagatggtgccggcggacgagctgaagcctacgcaggctgagttctcgccggagaaggtcgagcaggccaaggaagcgaatggcgaccgcgccgtgatcgtatccagcgacggccacatcatcgacggccaccatcaggcgctcgcggccaaggacgagggcaaggatgtcaaggcgatcgtgctggatgcgcccgtcgatcaggctctggaagccgtgaagaattctccgagcgcccagtacgagcagcctgccgaggccgcgccagcgcgtccgaagcgcctgctgggcgatgccaaggtcggcgacaccttcacgccatcgggcgatatcggctatgcctcgggcggccagacctaccgcgtggaatccgtcgaccgcaagggcggcatcaccgtgcgcaacgtggcgaccggcagcggcacgaccatctcgcgtgccgagttgatgggcgctgagcgcaagggcgtgacggtcgaaaagcagcaggccgacgccgcgccggccgatcagcccgccgcccccgccaatgaatcgttgttcgcaggcaacaagctcttcaccgaggacaaggttgaggcggcgcgcgcgcgcatgcgccgcaagctgaagtcgacggcgttcaccggcctggaccccgagctggtgacggacggcatgacgatcgccggcgcctacatcgaggccggcgtgcgcgagttcggcgcctatgccaaggccatggtcgaggacttcggcgccgaggtgaaaccctacctgctgtccttctacgaggcggtgcggcacttccctggcctggaaaccgagggtatgactgcgccccaggaggcaggccgcctgcatgcagagctgctgtctcagaacgaaacggcccaggaggcccaacatgacaccgaagcaaatccggcacgccctggagctggcgatgaaggacaaagcgccgcagcagtacgcggcgatgaaggcggacgggacgctggccgggtatctgaatcggctgacggcggacgtgcaggcgtcgcagagcgaggcgcacaacacgctggcgaacgatccgcagttccagaagatcagcgacccgcaaaccaggacgcaggaagcgagcagccggctgaaggcggccgagagcgtggcgctacagcaggcggtcgaggagatcagcagcctgcaagcgccgcagcaaaccaccgcatccgccccggcgacctgaagcgcgaagggtcgtggaaggacgcggccgagcgcaatgtgcagatcgtcgagctggtcaagcagctcgaaaaggacggccgcgtcgcaacgcccgatgagcgcgcgtcgctcgcccgctatacgggctggggtgcgtccgagatcgcaaacggcatcttcccggatcgcaacggccgctacaaggacgcgtcctggcaagcgctgggcgagcgcctgaaggcggcactcacgccggcggaatacgccgacgcgcgccgcacgatgcagtacgcccactacaccagcgagggggtgatccgcgcgatctacgcgggcctggatcgcctgggcttcaagggtggcgccatcctggagccgggcatgggcgtcggtcacttcaacggcctgatgccggcgcagatggccgcgcacagcagctacaccggcatcgagttcgacccgatcaccggcgccatcgcgcgcgcgctgtacccgcagagcaacgtgatcgtcggcgactacaccaagaccgcgctgccgaaggatttcttcgacgccgcgatcggcaacccgccgttcagctccaccgtcgtcagcaacgatgccgagtacaagaaacacggcttcatgctgcatgattacttctttgccaagacgctcgaccgcgtgaagccgggcggcgtcgtcgcgttcgtgaccagcaagggcacgatggacaaggccagcagccgcgcccgcgagttcatggccgagcgcgccaacctgatcggcgccgtccgcctgccgcagaccgcgttcaaggagaacgccggcaccgaagtcgtgaccgacgtgctgttcctgcaaaagcgcgggccgggcatccccgacaacggcgtgaagtggctggtcaccaaggaggtcgacacgtcgcagggcaagacgcctgtgaacgagtatttcgcagatcaccccgagatggtgctgggcgagcacgccaagaccggcagcatgtaccgcgccgacgagtacaccgtcgtgccgcgcgccggcgaggacatcgagcaggcattcgccaaggccatccagaacctgccggagggcggctaccgcccggcacgcggcagcgcggccgagcgtgctttcgtgcaggaccgcgatttcaacccgaaggccaagaaggagggcggcctgtacgtcagcgacgacggccgcctgatgcaggtcgaggacggcgcgggcgcggacctgacgcaccgcctcaactccgagggcaagcggatcgagctgaagccgaaggagcgagcgtggttgcgcggctgggtaagcctgcgcgacgcgctgaagcaggcgcagctcgaccagctcacggatggcgattggcagggatcgctccaggcgctcaacgccgcatacgacgcgttcgtcaaggatcacggcaacatcctggcgttctcgacgatcgagcgcgagaacccggacggcaccacgagcgtcacgcgccgcctgaagaacgcgccgctgatgacgatcgacgccgagggcgccctggcgcacgcgctggagcgcatcaacgaggacggcacgatcagcaagggcgcggtgctgcaagatcgcgttctgaagcgaccgaccgagccggtcatcaacaccacgcaagacgccatgttcgtggcgctcgaccgtcacggcaagttcgacctggacacggttgcgcgcttggcgggccagagccgcgagcaggccatcgccgacctgggcacggccgtctacgaggcgccgggcgccagctggcaaatggccgacgagtacctgtctggcaacgtggtgcgcaagctgaaggaggcccaggccgccgccgagatcgacaagcgctattcgcgcaacgtcgaggcgctgctggcggtgcagccgcgtccgctcggcccgcaggacatcagcgtcaagctgggcgccaattgggtgaagcccgccgacatcgagtcgtttgcccgcgaggcgctgggcgagcgcatggacgtcacctaccacgagaagctgaacgactggagcgtcgcgtcggtcgagcgtgctatcaccgagtggggtttcgacaagatgagcgcgtcgcagattctcgacgccacgctcaacaaccgccagatcaaggtcacgttccgcgaccaggacggcaagacgcacgtcgacatcgaggcgacgaccaaggccaacgacatctcgcagaagatgaaggacgccttccgccgttggatttggaccgacacggagcgcgccgaccgtctgacgcggtactacaacgagaacttcaacaacatcgcgccgcgcgcgtttgacggcagccacctgacgctgccgggcatgtcgtcgcgctacgccctgtacccgcaccagaagcgcgccgtgtggcgccaaattcaggcgggcgacacctacctcgcgcacgcagtgggcgccggcaagacgatggagatgatcgccggcggcatggagcagaagcgtctcggcctgatcgacaagccgatctacacggtgcccaatcacatgctggcgcagttcgcgcgcgagttcatggagctgtacccggccgcaaacatcatggtggccgacgaaacgaacttccacacgcacaaccgacgccgcttcatcgcccaggccgcgctcaacaaccccgacgccatcatcatgacgcactcgtcgtttggccgcgtcggcatgagcgaggagttcacgcgcagctacatcgagaagcagatcgccgagtggaaggaaattctcgacgagacggacaagagcgaccgcctgacccgcaagcaggtcgagcgccgcatcgagcagttggagcgccggatcgaggccaagcaggccaacgagaagaaggaccgcgtgctcacgttcgaggagctgggcgccgacatgctgttcgtcgacgagctgcacgagttccgcaagctcgacttcgccaccaaccagggcaacgtgaagggcatcgacccgtccggtagccagcgtgctttcgacctgatgatgaaggtcgaatacctgcgcagcaagaagccgggccgctcgctggtcggcgcgtcgggcacgcctgtgacaaacaccatgggcgagctgtacacggcgcagcgcctgttccagccgaagcagttggaagaggacggcctgacgtcgttcgacgcctgggccagccagtacggcgacatcgttgccggcttcgagcagaacgcggccggcggctatgaggtcgtgagccgcttcgccaagttccagaacgtgccggagctgatgcgccgcgtccgctccttcatggatatcctgaccagcagcagcctgggcgagctggtgaagcgcccgaccatcgagggcggccagcgcgaggtgcggatcacgcccgcgccggacggatataaagagtaccagcaggagctacagcagcgcattacggccatccgcgcgcgcaagggtccgccgaagaagggcgaggacatcatcttgcgcgtgatcggcgacggccggttctcggcaatcgacatgcgtttcgttgacccgtcgcggcgcaacgatcccggcagcaagctgaaccagatgatcgatgccgtgatcgaggactatcacgcgatggccgaccacgagtatttcgacacggccaccggcaagccggacccgatcaagggcgcgtcgcacctgattttcacggacatcggcctgggcgagcagtcggccgcgagccgtggcttcgacatgcgccgctggatcgagaagcgcctgacggacggcggcgtgccggccgaccacatcgcgttcatgcgcgactacaagcagcacgccaagaaggagcgtctgtttgccgacatgcgcgagggcaagaagcgcgtcctcatcggcggcaaggagatggagacgggcacgaacgtgcagaagcgcctcatgaccgaggatcacctggatgcgccgtggttcccggcttctgtcgagcaacgcgagggccgcatcgttcgccaaggcaaccagaacaagcaggtgcgcatccgcgcctgggcgacgaagggcagttacgacagcaccatgtggggcatgaacgcccgcaaggcgcgcttcatcgagcaggccatgaacggcgacgacaccgtgcgctcgctggaggacgtgagcgaggcgtcggcgttcgagatggccgccgccctggcctcgggcgacgagcgctacatgaagttggcgggcctgaagcaagacgtcgaccgcctggagcgcttgcgtcacgcgcactttggcgatcagcgccgcctctcggtcgagaagcaatacgctgtggatgccgtcgatcgcaacgagcgccgcatcgccgagctgacagaggcgatcaagcagcgcacgccgatccgcgcgggcgagtttctcgcgaaggccggcaaccgcgagttcgacgcgcgcgaggaattcagcgcggccctgttcgacgagttcaaggcgctgacaggcaaggagttcgacggcgagaaggtgatcggacacatcggtggcttcccgatcacctactacggcatgacgggccaaggctcgggcgcctacatcgcgggcgtgcacgtcggcatccctggcgaccccgacccgatcatcgcgtacccgatccgtggcgagttcgccatcaacggcatcgccgcgcgcgccgccaatcaggtcaacagcctggatcgcatggtggttgaggcgaaggatcggatcgagcagaaccagcgcaagatcaaccagatcgagagccgcctgggcgcgccgttccccgaggagggcgatctgctggagaaggtcgtcgcgctcaacaacctggagaacgagctgacggcggataaggcggccgagggcggcgagcaggtcagcgccgacgcggcggcggccacggtcgagacggaaggcggcgaggagggcccgcgctacagcgttgcgcctgaagtcgaccgcgatcagagcgtcgaagttacgagcatcaagacgttcgatgtgccgccggagcaattgtatgaaacggcaaaggctttctatcaagagaacctgcgcggcgtctccgtgaacaaccaatggatcggcaaggttgaattcaataaggagggcggcggcaagctactgtccgtagggaggcgcgagccgcttcacatgtcggttgttcaggcattgccgggacttctgaaaagcgccgtgcccgtccgattcgagcaggatcgaaagggccggcagcatatcgacggctactttgccgcgattgctcccgtggatattgatgggacgctctattccgtcgagctgaagttgcgcaaactcgacaagagcggaggtggccggacgacgttctacacccttgctgggttcgaggtagaaaaggtgggaggcggcagggcgaatccggagatagctaaccatggccggccttctaccgcctcccagccttctgagggttatgctgccgccgccgtctctaagcatggtactgcgacagcccctcgtcgggccttgactgttggagaattgctcgacgccgtgaattgggaccagcgttctttcagcatgtccccgtctccggcggggggcgaagtcaagtctcgccgacctgacgctatcgtaaatcaaacaatagcaaatgcaaacggcataaatgtaacggaattgtctatgacgttgcgaggaagcaacgtcggcgatctcatcggccgcctgctggaccgtggccgcgtggtgcttcacgatgacgcaggcagcctgccgggcaagaacgccccgcgcggcgtgcaggcttcgaccatgccggacggcaccatccacatggtgggcgccaacctgaacactcagagcgccgtgcccgtgctgctgcacgagatgttccatagcggcgtcaagccgctggtgggcgaggccgcatggggcaagctgatgacgcgcctggacgggctgctgcgccagtccgagcgctcgacgggccgcgcacgcgagttctacgacgccgcgttcaagcgcatgcgggcagcggcgcacgccggcgacgcgcgcgtgatgaacgccgaggagttcggcgcctacgcgatcgaggaatactccagcgcgccagccgccgtccgcgcctgggtcgacgacgtggtgggcgcgatcaaggcatggctgctgcgccgattgggcacgcagatcggcgccgtcacgccggcccagctccgcgccctggccgtggccgcgctgcgcagcgagccgcgcgccggcggcgacgcgccgcgcaactcggtgaaggcgccgatgaatccggtcgaggctggcctgacgccgccggcgcccacgcgcttcgatcgcttccaggcggcggtgcaggacaacatgaaccgcgtgaagcaggtgcaggagcgcatcaagaagctgaccggcctgaaggagcttggcttttccgactactaccgcgccgaggccaaccggcccggccgcgtggccgcgcgtctggaggatgcgcaaaagcacctgaccggcccgctgatggagcgccttgccaaatccggccacacgcaggcgcagctggaggaactgctgcacgcgcagcacgctgaggagcgcaacgagaagatcgccgagatcaacaaggacatgccggacggtggctctggcatgctgacggccgaagccaacgcgatcctggagaagtacaagggcgcgaccgagctgaaggccattgcgcagcaggcccgcgacatcgcgcgcgccacgctcgacatgaagctggcctacggcctgatcgaccagacgacgcatgacacgctgtcgaccggctacgagaactatgtgccgctgaagggcgacggcgagtatggcccgaagatcaagcgcgccatgggccacgaggagcgcgccgagcacatcctggagaacatcgcgcgcgactacgaccaagccgtcgcggccggcgagaagaacatcgctcgccagtcgcttctcgcgctggtggccgagcacgaagacccgagcctgtggacgataggcatcccgccgcgcgggcgccgcatcgccggtcaggtgttcaacgttgttgacccgagcctgcccaagggccagcagacgattggctcgttctccgcgcgctcgcaggtggacgcattcctggagggcgccggcccgaaggccgcaagctatctcgtgctggattccggcggcgagcgcgtgcagcagttcgtgaggccgttgcaggacaacgaggtgatggtctacgtcaagggcgagccggtgcgcatccagatttacgacgaggccctggcccgacagctccggccgctcgaccagagcaagatgcatccgatcctggagttcatgcgcggcatgaaccgctacctgtccaagatttacaccggctacaacccggctttcatcctgcgcaacgccgcgcgcgacgcactgaccggcacggtaaacatggtcggacacgaaggcgctggcgtcgccgcaaaagcctgggcgaagtatcccggcgcggtgaaggcgctgggccagtgggccgccaccggcaacgcgccggcgggcaagacgggcgagtacctgaaggagtaccgcatgcacggcggcaagactggcgcatcgtggatgtccgacctggagaccaagggcaaggagctgtcgcgcatgtacgaggacgcctacggcgcgagcggctacctgaaggacggccagaacctgaaggcggccaaggtcgccgggcgcaagatcgtcggcggcatggcgcatgtggtcgagatcgccaaccaagccacggagaacgcactgcgcctgtcgctgtacatgacgctgcgcgagagcggcgcatcgccgggcaaggccgcccaggccgccaagagcgtgacggtggactttgaccgcaagggcacgatgaccggcgcgctcggcgcggtctacctgttcctgaacccggcggtgcagggcacggccaacgccatgcgcacgctggcgagcggcgaacatcgcgggcaggcgttcgcggcgctcggcatgctggcgtcgctcggcttctacgcggcggcgtccggcatggacgacgacaaggaccgctggctgggcgagggctgggacgcgcgcaccaagaacttcgtgatgatgttcggcgaccacacactgaaggtcccgttgtcgcaggagttcacgccggcctatgctttcggcgtggcgctcgctgaggcgatgcgcggcgagggcgcgatgaaatcgtccgtccgcatggtctcgtcgttcctggatgcgtatttcccgctgaacggcgcctacagcccggacagcgacaatcacttgctcgatgcggctcaggccgcttcgccgacgatcatcaagccgctggttgataccgccgtgaaccgcaatcacttcggtagcccgatcgtgccggagacacccgcgaccaagagccagccggacaacctgaagatgtatcggggaacgaagggcacggtctacgacgctctggcgcagcagatcgcagcggcgggcgagctgaccggcgccggccgctacgagaacgacatcaccaagatcagccccgagacgctgaaaagcatatggcgcacgtacaccggcggtctcggccagttcgtgaccgactcgatcggcgcggccagcctcgccaccgagccgggcgagatgagcagcagcgacatcccgatcgcaaaggacttttggcgtgcccaggacgtcaagccgatccgcagccggtactacgatctcgcgcgcgaggcgaaggaagcagccacggaattccagcaggcgaagaaggccggcgacggcgaggcgatcgacagcatcttcgcgcggcccggccaagccgagttgatcgcgctcgacaagatgttcaagaagtcgagccacgccgccgcagcgatccgagatgaggaggtgacggtgaacgccgacaagtctctgtcgacgtccgagaagcgcaaccgcctgaaggagctggagaagcaggaggagtcgatctacagggatgcgctggacgtcttcaagacttccatcccttctaagtaggtgggttttgtgttgtagattgctcggagggagaggcttagcgaatggcaaagcttccccatacccagcagggtgtagaaagcttgccatgtttgctctggcggagccggcttgagcattcaggcaccacagtctatcgacacccgctgtggctactccagacttcgacgacggtgcgagtcgtccatcccgggctctagagctttcgcttcccaccctttgcgcttgggccttcctgcgctacgccgcgtgcggaacgtttggataccgatagcttccgcgacggctggacgtgaaaaagcccggtgattcattgttgggcgggcctttggcaaggcaaccggcgcacgccccaaaacacgcgcgctgattacaccgcccaacaatggataccgggcttggtgatgctttttttggggttgcgctggaagccaatccagcgaaaccatcatagcacaagaagtcgatgcgtgcgaattttatacgcagtggccgctgacaaccgcagagcatccttctccgtcataggcagtcgggcgcgggttgtcatcgcgaagagcatgaccaagcatgatgaagagcgcggccacgatggcgagcacgacggtgatgtatgtcttcgggaagtgtccgcaggcccagctgtcgaattgcgagtagcgctcggcgatcttcttggcgatgtgcgaaagcttttccatgatcttcgaatttattggttgtgagttcgtattctaggtgaattatttcttcctgtgtcgaattttctcctcgtcgtcgagctggcgcatgtaggcgcgcaccttgcggaatcgttcgatctcgcgggtgcacgccgcatccacggcggcgcggatgaaggcggccttggagaagatcaccttgttgtcgatcaggtactccatttgcaggatcgtcggctcggtgaagcgcacgttgatgaagacgttgcgcgtcgggtcgccatgtgaccatggcgcctcatccttggatgccgttgtcttggtcgccgcgcgccgcgcacgatcgccgcgccgtggtttggtgttcgccatgcttcctccgatgcgcgccgcaatgaatctggagcgctctttatcactataggatcggtgcggattggtgcgatgcgattttgcgcatgcctgtgcagatagcggtttgaaaatcgttgcttcggtaataaaatcggaccagccgaagcaggagattttgtcaatgccccaaactagcaagcaactcaccgccgacgactatgcccgcgcggctaccgcgctaggcgtgccggtcgctgccgtaaaggcggtgacggaggtggagagcaacggcaagggcttcctgccggatggccgcccactgatcttgtacgagcgccacatcatgcggcgccagctcgtcgcggcaggtcacgcgatggatgccacgcactacaacttgaccgacccgaacatcgtcaactcgaagcccggcggctacgtcggtggtgctggagaatgggatcgtctggcacgcgcgatcgagatcaaccggccggcggcactggaatcggcgagttggggcctgttccagatcatgggatttcactggaagctgctgggcttcgcgtcggcgcaggcgttcgtcaacgccatgtacacgagcgagggcgcgcagctcgacgcgttcgtcgccttcgtgaaggccaacccgaacctgctgcgcgcgctccgtgccaagaactggccggacttcgcgcgcggctacaatgggccggcctacgagaaaaatcactatcacatcaagctggctgaagcgtatgcaaagcacgcatgatctcgtgaagcacatccttgaactcgttcgccggcgtatcgccacgagcgcgatttgcgaagttcgtcgcgagaacgatgttgccgattgtgtagccaagcttcggcgccagacggtcgatggacggcttcattggatgccgagctgggccggtcatgctgaattcagcaccggtccagtagcagcggcgctcttgccgagcgagaagccaaatgcagtattccatcgtgatgtcgcacggcatccccgcttcctgactgcgtttcttcgcgccggccacgagtccgcttatgatgccctcgtcggacacattccagcgctcgcgcgcggcgcggcgcagttccttcgcgcgcgcggtcgcctcgtattggcgccccttctcgcgagcgcgatcgatgttttcgtttttccacacgagcgcgttgaggcgcatgcagatcatgcagtttccgtgcgtcttcccgcgaacggattgctcggcacctccgtacgcgtgcccgcgcgcgcaaagcgggccaagatagtgcttcgactcgtcgatggagatagcgagggtggatgccggaaaccggccttccgcaaggtactgatcgatcttatcggcggttttcataatcctttctggcataacggactgaccagaattgtagcatgtccgttgtaaccaaacgggagccgcatgaacctcatcgaaaacgccggccagtggcacaagctctggtcgatccgctttgcgctgctgagcgccatgctcgcggcggccgaggcggccctgccgctgtggaatggcatcgtgccgccgcacgtctttgcatcgatctcgacgctgtgcggaatcgcttcggcggtatcgcgcgtggtgcagcaaacggcgctccgggccggcgacggcgccgacccgcagtgaagacctcccgcccatgcctcacggtcatcggccgatcatcacaccccttcttcctggggtggtgatgcaacggtcaacgacgagggtggcacatgaacacgacgacatggacagagatgaatccgccggatagccaactgtgggggttgatcggcggcgcggctggcgcggtggtcgccgcgttcgtcatgctgcgcaagtacctgtcccgcgatgcggtcgatcgcgctggcgacgccgccgacatcggcgccatcaagcgcctgaacgacctgctggacgaggagcgcaaggcgcgacgcgaggccgaagcgcgtgccgacgtgttcgccaaggagcgcaacgaggcgatcatgcagatcggcgagctgaaagggcagatcgcggcgctgaccatgcaggtcgcgctgctgaacagcaaggttgagcaatatgcaaactcccaggttgctgcgcctcaaggaaatccttgagcgcttctatgccgcgcgcaagcgatccttcaacgagatcactgtgtggtcgctgatcacgctgctggcggtcagcagcggatcggttggctacttcctaggcgacttgcagaacaacgtcgagctggccgccacgcgcgcgcagcatcagcgcgatcttgaccacctgcgcgcagcctacgaggcggccatggccgagcgcgatgcgcgcctggacgatctggcgaggcaggtggcgcaggcggcccgcacatcgcagcatgccgccactacggccgagcgcgccgcgaccgcgccgaaagccgaatccccgacgcgccgcccgcagggcgtgccgtatctgccgcaactgcctccgtcgctgcacaagggtggatgatgaacctgatcaactggaagggctacgccattgccgccggcgccggcgcgctggtcgccgcgatcctggccggctggggcgcctgggcgatccaggccaaccggtacgatgtgcagatcgcagagctgaagcgcgtgcacgccgagcagttgggcgagatcgccgcgaagggcgccgagcaggcccgcgaggcgctggattggaagggccgggcagagaaggcaaccgccgagatcgatgcccgacaacaggagaacgatcgtgtcaaagctgaaaacgctagtcttgctgacgagctgcgcactggcgctcgccgcgtgttcgtccgtgccaagtgccccgctgccggtggtggcgcagtgcccggagccgccgccgccgcccgcgtggacgatgagggcgcgcgagccgaacttgaccctgcgtttgcggggtctctggcaggaatcaccgacgacggcgacgacgccatccgtgaactgaccgccttgcaggactacgtgcgacgcgtctgtttagcgccgcgaaactagcgcatcagccgcacatggcataatatgcaagttacattaggggattatg

(19) United States
(12) Patent Application Publication
(10) Pub. No.: US 2024/0164387 A1
(43) Pub. Date: May 23, 2024

(54) BACTERIOPHAGE, BACTERIAL WILT DISEASE CONTROL AGENT, AND BACTERIAL WILT DISEASE CONTROL METHOD

(71) Applicant: PANEFRI INDUSTRIAL CO., LTD., Nagaokakyo-shi (JP)

(72) Inventors: Toshiaki HARASHIMA, Okinawa (JP); Kazuma NAKANO, Okinawa (JP); Toru KARIMATA, Okinawa (JP)

(73) Assignee: PANEFRI INDUSTRIAL CO., LTD., Nagaokakyo-shi (JP)

(21) Appl. No.: 18/007,441

(22) PCT Filed: Jul. 30, 2020
(86) PCT No.: PCT/JP2020/029188

§ 371 Date: Jan. 30, 2023

(51) Int. Cl. A01N 63/40; A01P 1/00; C12N 7/00; C12N 9/36; C12N 15/52

(52) U.S. Cl. CPC A01N 63/40; A01P 1/00; C12N 7/00; C12N 9/2462; C12N 15/52; C12N 2795/10243

(57) ABSTRACT

A bacteriophage comprising a gene encoding lysozyme fused-type DarB, having genes encoding three lytic enzymes of an endolysin, the DarB and Rz/Rz1, having a gene encoding CsrA, having a gene encoding two different tail fiber proteins and infecting Ralstonia solanacearum, a bacterial wilt disease control agent comprising the bacteriophage as an active component and a bacterial wilt disease control method in a plant comprising administering the agent to a plant or a plant growth medium, for providing a bacteriophage which is effective against a wide variety of Ralstonia solanacearum strains and a bacterial wilt disease control method using such bacteriophage.

### CROSS-REFERENCE TO RELATED APPLICATIONS

The present application is the national stage of international application PCT/JP2020/029188, filed on Jul. 30, 2020.

### TECHNICAL FIELD

The present invention relates to a bacteriophage, a bacterial wilt disease control agent and a bacterial wilt disease control method.

### BACKGROUND ART

Ralstonia solanacearum infects 200 plant species or more such as the Solanaceae plants and causes bacterial wilt. When bacterial wilt progresses, the plant infected with Ralstonia solanacearum withers.

Major chemical pesticides which have been used so far against bacterial wilt are chloropicrin and bromomethane, which are powerful drugs.

In view of the problems such as the increase in the effective application amounts, the environmental pollution, the ozone layer depletion, the influences on the health and the pesticide residues, however, development of safe alternative pesticides and control techniques which replace the chemical pesticides is desired.

Although techniques using bacteriophages (PTLs 1 to 3) have been known so far as safe alternative pesticides and control techniques which replace the chemical pesticides for example, the techniques are effective against a limited number of Ralstonia solanacearum strains, and a technique which is effective against a wider variety of Ralstonia solanacearum strains has been desired.

### CITATION LIST

### Patent Literature

PTL 1: JP-A-2007-252351

PTL 2: JP-A-2018-24589

PTL 3: WO2017/104347

### SUMMARY OF INVENTION

### Technical Problem

Accordingly, a problem of the invention is to provide a bacteriophage which is effective against a wider variety of Ralstonia solanacearum strains and a bacterial wilt disease control method using the bacteriophage.

### Solution to Problem

As a result of extensive studies to solve the problem, the present inventors have found that a bacteriophage having specific genes infects a wide variety of Ralstonia solanacearum and thus have completed the invention.

That is, the invention is a bacteriophage characterized by
having a gene encoding lysozyme fused DarB,
having genes encoding three lytic enzymes of an endolysin, the DarB and Rz/Rz1,
having a gene encoding CsrA,
having genes encoding two tail fiber proteins and
infecting Ralstonia solanacearum.

Moreover, the invention is a bacterial wilt disease control agent characterized by containing the bacteriophage as an active component.

Furthermore, the invention is a bacterial wilt disease control method in a plant characterized by administering the bacterial wilt disease control agent to a plant or a plant growth medium.

### Advantageous Effects of Invention

The bacteriophage of the invention infects a wide variety of Ralstonia solanacearum strains and thus is effective for controlling bacterial wilt.

### BRIEF DESCRIPTION OF DRAWINGS

FIG. 1 shows an example of the appearance of the bacteriophage RKP180 of the invention (the length of the bar in the figure is 20 nm).

FIG. 2 shows a phylogenetic tree (Maximum Likelihood) based on the whole genome sequence of RKP180.

FIG. 3 shows a phylogenetic tree (Maximum LikeLihood) based on the amino acid sequence of a large terminase of RKP180.

FIG. 4 shows the genetic map of RKP180.

FIG. 5 shows the genetic maps of RKP180 and related strains thereof.

FIG. 6 shows a figure comparing the whole genome identities of RKP180 and related strains thereof, GP4 and Bcep22.

FIG. 7 shows the functional domains of the DarB gene products of RKP180 and GP4.

FIG. 8 shows an alignment of the endolysins of RKP180 and GP4.

FIG. 9 shows one infection cycle of RKP180 (mean±SD (N=3)).

FIG. 10 shows the control effect of RKP180 on bacterial wilt.

### DESCRIPTION OF EMBODIMENTS

The bacteriophage of the invention has a gene encoding lysozyme fused DarB, has genes encoding three lytic enzymes of an endolysin, the DarB and Rz/Rz1, has a gene encoding CsrA, has genes encoding two tail fiber proteins and infects Ralstonia solanacearum.

The gene encoding lysozyme fused DarB encodes a DarB protein fused with lysozyme unlike the conventional Dar-encoding gene. Lysozyme has bacteriolytic activity and has bactericidal action. DarB has a DNA methylase domain and a helicase domain and has a defense function for protecting the own DNA from the restriction mechanism of the host bacterium (“DarB” is sometimes called “restriction mechanism defense gene DarB” or the like below). In many cases, lysozyme and DarB protein are encoded by individual genes, but the DarB of the bacteriophage of the invention is a rare DarB protein in which the two are fused. Due to the lysozyme fused-type DarB protein in which lysozyme is fused, the bacteriophage of the invention can avoid the restriction mechanism of the host bacterium, replicate the own genetic information efficiently in the host, lyse the host bacterium efficiently and grow efficiently. Examples of such a fused gene include gp70 (YP_002922742.1) of Burkholderia virus BcepIL02, gp75 (NP_944303.1) of Burkholderia virus Bcep22, gp65 (YP_006589997.1) of Burkholderia virus DC1, the gene from the 44,008th to the 57,543rd residues (ORF66) of the base sequence of SEQ ID NO: 1 and the like. Of the genes encoding lysozyme fused DarB, the gene from the 44,008th to the 57,543rd residues (ORF66) of the base sequence of SEQ ID NO: 1 is preferable.

In the invention, as long as the DarB maintains the properties, genes having a sequence identity of 50% or more, preferably 55% or more, to any of the genes encoding lysozyme fused-type DarB are also included in the genes encoding lysozyme fused-type DarB.

The genes encoding three lytic enzymes are an endolysin, the DarB and Rz/Rz1. Because harboring more than one lytic enzyme results in increase in bacteriolytic efficiency, bacteriolytic efficiency of the bacteriophage of the invention also increases. The gene encoding an endolysin has lysozyme-like lytic enzyme activity with N-acetylmuramidase activity. Examples of such gene include ORF65 (AXG67760.1) of Ralstonia phage GP4, Cupriavidus oxalaticus WP_06323822?0.1, Cupriavidus taiwanensis WP_115662684.1, Ralstonia mannitolilytica WP_115075602.1, the gene from the 58,762nd to the 59,355th residues (ORF69) of the base sequence of SEQ ID NO: 1 and the like. Of the genes encoding an endolysin, the gene from the 58,762nd to the 59,355th residues (ORF69) of the base sequence of SEQ ID NO: 1 is preferable.

In the invention, as long as the properties of the endolysin are maintained, genes having a sequence identity of 50% or more, preferably 80% or more, to any of the genes are also included in the genes encoding an endolysin.

The DarB gene which is one of the genes encoding three lytic enzymes mentioned above encodes the lysozyme fused-type DarB.

Of the genes encoding three lytic enzymes, the Rz/Rz1 genes encode spanin proteins which degrade the outer membrane of a bacterium. The Rz gene and the Rz1 gene overlap each other, and the Rz1 gene encoding the Rz1 protein is in the Rz gene with a different reading frame. Examples of the gene like Rz include ORF61 (AXG67756.1) of Ralstonia phage GP4, gp80 (NP_944308.1) of Burkholderia virus Bcep22, gp72 (YP_006590002.1) of Burkholderia virus DC1, WP_090835650.1 of Paraburkholderia hospita, the gene from the 61,114th to the 61,647th residues (ORF74) of the base sequence of SEQ ID NO: 1 and the like. Of the genes encoding Rz, the gene from the 61,114th to the 61,647th residues (ORF74) of the base sequence of SEQ ID NO: 1 is preferable. In the invention, as long as the properties of Rz are maintained, genes having a sequence identity of 30% or more, preferably 90% or more, to any of the genes are also included in the genes encoding Rz.

Moreover, examples of the gene like Rz1 include QIQ65891.1 of Pseudomonas phage Epa38, ASZ72374.1 of Pseudomonas phage vB_PaeS_S218, CRM53430.1 of Pseudomonas sp. 31R17, gp73 (YP_006590003.1) of Burkholderia virus DC1, gp81 (YP_009173772.1) of Burkholderia virus Bcep22, the gene from the 61,352nd to the 61,597th residues (ORF75) of the base sequence of SEQ ID NO: 1 and the like. Of the genes encoding Rz1, the gene from the 61,352nd to the 61,597th residues (ORF75) of the base sequence of SEQ ID NO: 1 is preferable. In the invention, as long as the properties of Rz1 are maintained, genes having a sequence identity of 30% or more, preferably 45% or more, to any of the genes are also included in the genes encoding Rz1.

Many bacteriophages having any of the genes encoding the three lytic enzymes are known, but no bacteriophage having all of the three lytic enzymes having a sequence identity of 80% or more can be found in known databases. It is believed that the bacteriophage of the invention lyses a bacterium efficiently because the bacteriophage has all the three lytic enzymes.

The gene encoding CsrA is a gene encoding CsrA which suppresses biofilm formation. Examples of such gene include ORF4 (AXG67699.1) of Ralstonia phage GP4, WP_066491652.1 of Burkholderia sp. BDU8, CsrA (WP_034473805.1) of Caballeronia zhejiangensis, gp46 (YP_006589976.1) of Burkholderia virus DC1, gp49 (YP_002922721.1) of Burkholderia virus BcepILO2, gp54 (NP_944283.1) of Burkholderia virus Bcep22, the gene from the 28,351st to the 28,548th residues (ORF47) of the base sequence of SEQ ID NO: 1 and the like. Of the genes encoding CsrA, the gene from the 28,351st to the 28,548th residues (ORF47) of the base sequence of SEQ ID NO: 1 is preferable. In the invention, as long as the properties of CsrA are maintained, genes having a sequence identity of 50% or more, preferably 95% or more, to any of the genes are also included in the genes encoding CsrA.

The gene encoding tail fiber proteins is a gene(s) encoding a tail fiber protein(s) having a receptor binding domain related to the host recognition/binding at the C-terminus. Examples of such gene include gp75 (AXG67770.1), gp76 (AXG67771.1) and gp77 (AXG67770.1) of Ralstonia phage GP4, gp36 (YP_009218128.1) of Ralstonia phage RSJ5, Burkholderia virus DC1 gp56 (YP_006589986.1), gp57 (YP_006589987.1), gp59 (YP_006589989.1) and gp60 (YP_006589990.1), gp64 (NP 944293.2), gp65 (NP 944294.2) and gp66 (NP 944295.2) of Burkholderia virus Bcep22, gp60 (YP_007236806.1), gp61 (YP_007236807.1) and gp62 (YP_007236808.1) of Burkholderia virus Bcepmigl, gp58 (YP_002922730.1), gp59 (YP_002922731.1), gp61 (YP_002922733.1) and gp62 (YP_002922734.1) of Burkholderia virus BcepILO2, the genes from the 36,001st to the 37,320th residues (ORF58) of the base sequence of SEQ ID NO: 1 and from the 37,406th to the 38,278th residues (ORF59) of the base sequence of SEQ ID NO: 1 and the like. In the invention, one or two of the genes encoding tail fiber proteins may be contained, but the different two genes from the 36,001st to the 37,320th residues (ORF58) and from the 37,406th to the 38,278th residues (ORF59) of the base sequence of SEQ ID NO: 1 are preferable. In the invention, as long as the properties of the tail fiber proteins are maintained, genes having a sequence identity of 10% or more, preferably 85% or more, to any of the genes are also included in the genes encoding tail fiber proteins. The bacteriophage of the invention has the two different genes encoding tail fiber proteins and thus has a wide host range.

As described above, the bacteriophage of the invention having the genes encoding three lytic enzymes of endolysin, DarB and Rz/Rz1, the gene encoding lysozyme fused-type DarB, which is a defense gene against the restriction mechanism of the host bacterium, and the gene encoding two tail fiber proteins for a wide host range infects a wide variety of Ralstonia solanacearum strains with an efficient bacteriolytic cycle. Moreover, the bacteriophage has the gene encoding CsrA, which suppresses biofilm formation, and thus exhibits a stronger control property.

The Ralstonia solanacearum infected with the bacteriophage of the invention is not particularly limited as long as the Ralstonia solanacearum is any of those found from 200 plant species or more such as the Solanaceae plants, the Zingiberaceae plants and the Lamiaceae plants, but the bacteriophage infects at least all of strain MAFF107624, strain MAFF211266, strain MAFF211270, strain MAFF211543, strain MAFF301859, strain MAFF311644, strain MAFF730103, strain MAFF730131, strain MAFF302745, strain MAFF311632, strain MAFF211536, strain MAFF331041, strain MAFF730139, strain MAFF211280, strain MAFF211533, strain MAFF211468, strain MAFF211516, strain MAFF311101, strain MAFF311102, strain MAFF211479, strain MAFF211471, strain MAFF211483, strain MAFF211484, strain MAFF211486, strain MAFF211272, strain MAFF211276, strain MAFF211278, strain MAFF211490, strain MAFF211492, strain MAFF211497, strain MAFF211476, strain MAFF211414, strain MAFF211429 and strain MAFF301558 and further infects the 70 Ralstonia solanacearum strains or more collected by the present inventors. In this regard, the Ralstonia solanacearum strains having a number with MAFF can be obtained from Genebank Project, NARO, National Agriculture and Food Research Organization.

In this regard, the infection here refers to the state in which plaques are formed on an agar plate on which Ralstonia solanacearum grows or the state of lysing in a liquid medium in the presence of the bacteriophage of the invention.

The bacteriophage of the invention described above can be obtained by screening known bacteriophages according to a general method to find those having all the above genes and infecting Ralstonia solanacearum.

Moreover, the bacteriophage of the invention should have the above genes. Thus, the bacteriophage of the invention may belong to any group, any order or any family but preferably belongs to the family Podoviridae of the order Caudovirales of the group I.

The bacteriophage of the invention has a head diameter of 40 to 90 nm, preferably 70 to 80 nm, a tail length of 5 to 30 nm, preferably 15 to 25 nm and a width of 5 to 20 nm, preferably 9 to 15 nm.

The bacteriophage of the invention has a double-stranded genome.

The genome size of the bacteriophage of the invention is not particularly limited but is for example 6,000 to 280,000 bp, preferably 50,000 to 70,000 bp, more preferably 61,000 to 63,000 bp. The genome size is the value of the whole genome base sequence.

The GC content of the bacteriophage of the invention is not particularly limited but is for example 55 to 75%, preferably 60 to 70%, more preferably 63 to 66%. The GC content is the value calculated from the whole genome base sequence.

The number of genes in the bacteriophage of the invention is not particularly limited but is for example 10 to 330 genes, preferably 60 to 80 genes, more preferably 76 genes. The 76 genes include 75 open reading frames (ORFs) and one tRNA.

The genomic DNA of the bacteriophage of the invention is digested preferably into five or more fragments through treatment with HindIII restriction enzyme. More preferably, the number of fragments of 600 bp or more is five or more, and particularly preferably, the number of fragments of 600 bp or more is five. The conditions for treatment with HindIII restriction enzyme are at 35 to 40° C. for 12 to 24 hours.

Furthermore, the bacteriophage of the invention may have, for example, genes such as tRNA-Ser and lysogenic repressor (ORF9) and the like.

A preferable example of the bacteriophage of the invention explained above is RKP180 found by the present inventors. RKP180 was deposited for an international deposit at NITE Patent Microorganisms Depositary, National Institute of Technology and Evaluation (address: #122, 2-5-8 Kazusakamatari, Kisarazu-shi, Chiba 292-0818, Japan) on Mar. 27, 2020 under NITE BP-03185.

The bacteriophage of the invention also includes those having modification, mutation or the like while maintaining the properties.

Because the bacteriophage of the invention can infect a wide variety of Ralstonia solanacearum, a bacterial wilt disease control agent can be obtained using the bacteriophage as an active component.

In this regard, the control here refers to suppression and alleviation of infection of a plant with Ralstonia solanacearum, suppression and alleviation of the onset of bacterial wilt, suppression and alleviation of spread of infection with the disease or control of Ralstonia solanacearum.

The amount of the bacteriophage of the invention in the bacterial wilt disease control agent may be appropriately set depending on the purpose of control, but for example, the bacteriophage of the invention may be contained at potency of 10³ to 10¹² pfu/mL, preferably 10⁵ to 10¹¹ pfu/mL.

The bacterial wilt disease control agent may contain only the bacteriophage of the invention as an active component, but may further contain another substance, a composition or the like such as a pharmacologically and agronomically acceptable protein stabilizer.

The bacterial wilt disease control agent described above can control bacterial wilt in a plant when administered to a plant or a plant growth medium. Examples of the plant include tomatoes, eggplants, bell peppers and tobaccos of the family Solanaceae, Perilla frutescens var. crispa and Perilla frutescens of the family Lamiaceae, ginger, turmeric and curcuma of the family Zingiberaceae and the like. The plant growth medium may be soil, a solid medium such as a mat containing organic materials, a liquid containing a nutrient solution or the like. The methods and the conditions for administering the bacterial wilt disease control agent to such a plant or a plant growth medium are not particularly limited but are, for example, spraying or dropping to the plant or the plant growth medium, injection into the plant or the like.

### EXAMPLES

The invention is explained in detail below referring to Examples, but the invention is not limited to the Examples.

### Example 1

### Separation of Bacteriophage:

Soil was suspended in water and then left still, and thus a supernatant was obtained. The supernatant was filtered with a 0.25 μm filter, and the filtrate was subjected to a plaque assay using Ralstonia solanacearum as the host. By isolating a formed plaque, bacteriophage RKP180 was obtained.

### Example 2

### Analysis of Bacteriophage RKP180:

### (1) Electron Microscope Analysis

A phage suspension of 4×10¹⁰ pfu/mL was stained with 1 uranyl acetate, and pictures were taken under an electron microscope (JEM-1400Pus, manufactured by JEOL Ltd.).

### (2) Determination of Genome Size

The genomic DNA was purified from the phage particles by phenol/chloroform extraction. The purified genomic DNA was subjected to agarose gel electrophoresis with Mupid-2plus electrophoresis apparatus (manufactured by Mupid) using 0.3% agarose (Agarose 1200 Standard Type, manufactured by PH Japan Co., Ltd).

According to the bands of a size marker, the genome size of RKP180 was 48,500 bp or more.

### (3) Determination of Genomic Structure

The purified genomic DNA was treated with a single-stranded DNA nuclease (Si Nuclease, manufactured by Promega Corporation), a DNA nuclease (Recombinant DNase I, manufactured by Takara Bio Inc.) and an RNA nuclease (RNase A, manufactured by Takara Bio Inc.) and subjected to electrophoresis by the above method.

Because the DNA of RKP180 was not digested with the single-stranded DNA nuclease and the RNA nuclease but was digested with the DNA nuclease, it was found that the genome of RKP180 is double-stranded DNA.

It was found that the genomic DNA of RKP180 is digested into five fragments of 600 bp or more through treatment at 37° C. for 16 hours using HindIII restriction enzyme.

### (4) Genomic Analysis

The genomic DNA sequence of RKP180 was determined by shotgun sequencing using PacBio RS II (manufactured by PACIFIC BIOSCIENCES). The determined base sequences were assembled using RS_HGAP Assembly 2.3.0. The identity search of the whole genome sequence to calculate the most closely related strain was conducted with BLASTN. The open reading frames (ORFs) were determined with MetaGeneAnnotator 1.0 of MiGAP ver2.23 (Database Center for Life Science), tRNAscan-SE 1.23, BLAST 2.2.18 and RNAmmer 1.2, and homology search was conducted using BLASTP and PSI-BLAST against the COG, Refseq, TrEMBL and nr databases. In the homology search, as the cut-off for the similarity, E-value was set to less than 1e-4. For the ORF domain search, BLASTP and InterPro 78.1 were used. For the alignment of amino acid sequences, Clustal2.1 was used. The genetic maps were created using the data obtained with MiGAP. The phylogenetic trees of the whole genome sequences and the amino acid sequences of large terminase were created by the maximum likelihood method after identity comparison with ClustalW of MEGA 7.0.26. The genome sequences of Ralstonia phage RSB2 (AB597179.1), Ralstonia phage GP4 (MH638294.1) and Burkholderia phage Bcep22 (AY349011.3) of PTL JP-A-2018-24589 were obtained from NCBI, and the annotation of RSB2, GP4 and Bcep22 was conducted with MiGAP by the above method.

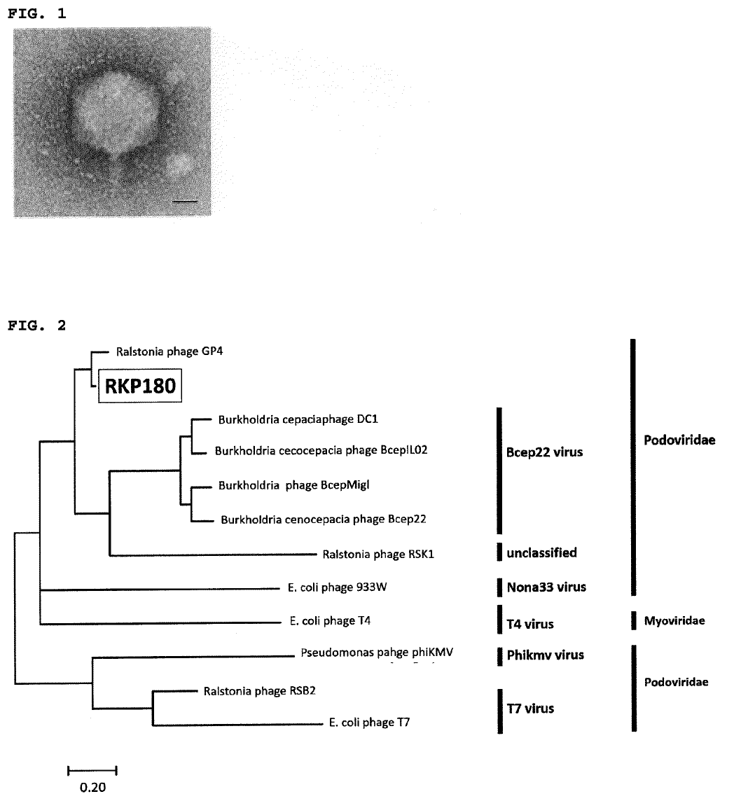

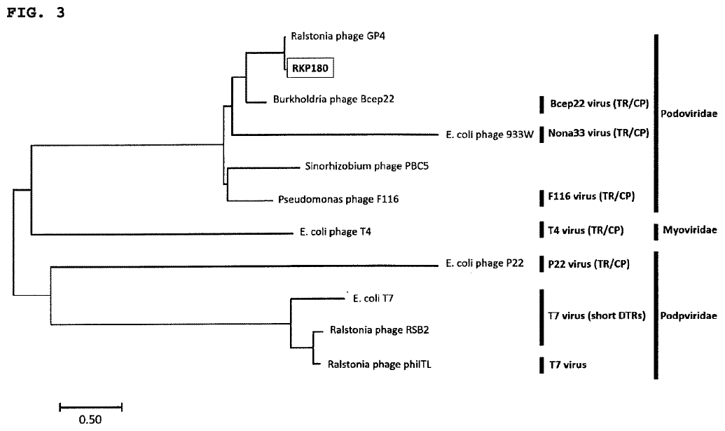

The genome of RKP180 was linear double-stranded DNA. The genome size was 61,696 bp, and the GC content was 64.6%. The whole genome of RKP180 which was determined as described above is shown in SEQ ID NO: 1. From the shotgun sequencing using PacBio RS II and the phylogenetic analysis based on the entire base sequence, it was shown that RKP180 genome belongs to the genus Bcep22-like virus of the family Podoviridae (podovirus) of the order Caudovirales, which is consistent with the results of the electron microscope analysis (FIG. 1, see below). As a result of the homology search with BLASTN, the most closely related strain was known Ralstonia phage strain GP4 (coverage of 77% and identity of 97%, Table 1 and FIG. 2), and the next related strains were Burkholderia cenocepacia phage strain BcepIL02 (coverage of 20% and identity of 78%), Burkholderia phage strain DC1 (coverage of 21% and identity of 78%), Burkholderia cepacia phage strain Bcep22 (coverage of 22% and identity of 78%) and Burkholderia phage strain BcepMigl (coverage of 21% and identity of 78%) (Table 1 and FIG. 2).

TABLE 1

| | | | | | | | | Base | | |
|---|---|---|---|---|---|---|---|---|---|---|
| | | | | | | | | Length | GC | Gene |
| Top | Accession | Species | Coverage | Identity | Family | Genus | Structure | (b) | (%) | Number |
| 1 | MH638294.1 | Ralstonia | 77% | 97% | Podoviridae | — | Linear | 61,129 | 64.0 | 83 |
| | | phage GP4 | | | | | | | | |
| 2 | FJ937737.2 | Burkholderia | 20% | 78% | Podoviridae | Bcep22 | Linear | 62,715 | 66.2 | 77 |
| | | cenocepacia | | | | | | | | |
| | | phage | | | | | | | | |
| | | BcepIL02 | | | | | | | | |
| 3 | JN662425.1 | Burkholderia | 21% | 78% | Podoviridae | Bcep22 | Linear | 61,847 | 66.2 | 73 |
| | | phage DC1 | | | | | | | | |
| 4 | AY349011.3 | Burkholderia | 22% | 78% | Podoviridae | Bcep22 | Linear | 63,882 | 65.3 | 78 |
| | | cepacia | | | | | | | | |
| | | phage Bcep22 | | | | | | | | |
| 5 | JX104231.1 | Burkholderia | 21% | 78% | Podoviridae | Bcep22 | Linear | 62,952 | 65.5 | 76 |
| | | phage | | | | | | | | |
| | | BcepMigl | | | | | | | | |
| 6 | CP013389.1 | Burkholderia | 15% | 0 | Burkholderiaceae | Burkholderia | Cyclic | 4,439,942 | 66.3 | 4,064 |
| | | sp. BDU8 | | | | | | | | |
| | | chromosome 1, | | | | | | | | |
| | | complete sequence | | | | | | | | |
| 7 | MH252365.1 | Ralstonia phage | 2% | 0 | Myoviridae | Jilinvirus | Linear | 44,526 | 62.3 | 24 |
| | | phiRSP, | | | | | | | | |
| | | complete genome | | | | | | | | |
| 8 | AB863625.1 | Ralstonia phage | 6% | 0 | Podoviridae | T7 like | Linear | 40,471 | 59.6 | 54 |
| | | RSK1 DNA, | | | | | | | | |
| | | complete genome | | | | | | | | |

Moreover, from the phylogenetic analysis based on the amino acid sequence of large terminase, the genomic DNA of RKP180 was classified into the clade having a circular permuted [CP] structure with a terminal redundancy [TR] sequence (FIG. 3).

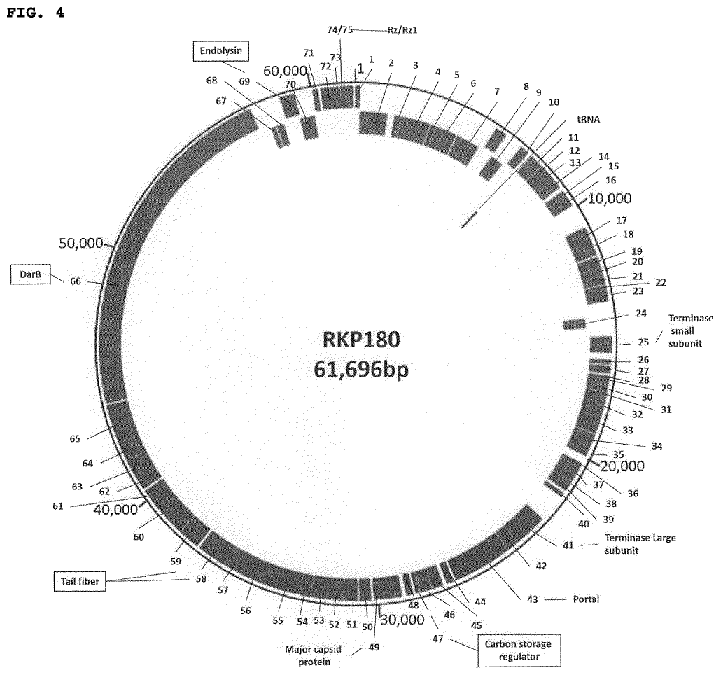

As shown in FIG. 4, the genome of RKP180 comprised 76 genes in total, which encoded 75 ORFs and one tRNA. A list of the functions and the products of the genes is shown in Table 2.

TABLE 2

| | Start | End | Length | Length | | | |
|---|---|---|---|---|---|---|---|
| ORF | (nt) | (nt) | (nt) | (aa) | strand | putative function | organism |
| 1 | 1 | 201 | 201 | 66 | + | Na+-driven multidrug | Ralstonia |
| | | | | | | efflux pump | phage GP4 |
| 2 | 191 | 1,393 | 1,203 | 400 | − | Integrase | Ralstonia |
| | | | | | | | phage GP4 |
| 3 | 1,695 | 1,964 | 270 | 89 | − | Pyocin activator protein | Rhizobium sp. |
| | | | | | | | Root483D2 |
| 4 | 1,976 | 3,238 | 1,263 | 420 | − | Fructose-2,6-bisphosphatase | Ralstonia |
| | | | | | | | phage GP4 |
| 5 | 3,278 | 3,514 | 237 | 78 | − | DNA-binding transcriptional | Ralstonia |
| | | | | | | regulator | phage GP4 |
| 6 | 3,511 | 4,467 | 957 | 318 | − | Recombinational DNA repair | Ralstonia |
| | | | | | | protein RecT | phage GP4 |
| 7 | 4,509 | 5,537 | 1,029 | 342 | − | Phage-related protein, | Ralstonia |
| | | | | | | predicted endonuclease | phage GP4 |
| 8 | 5,729 | 6,208 | 480 | 159 | + | HNH endonuclease and | Ralstonia |
| | | | | | | DNA-binding | phage GP4 |
| 9 | 6,209 | 6,799 | 591 | 196 | − | Transcriptional regulator | Ralstonia |
| | | | | | | | phage GP4 |
| 10 | 6,928 | 7,296 | 369 | 122 | + | DNA-binding transcriptional | Ralstonia |
| | | | | | | regulator | phage GP4 |
| tRNA | 7,302 | 7,388 | 87 | 28 | + | NA | Ralstonia |
| | | | | | | | phage GP4 |
| 11 | 7,453 | 7,980 | 528 | 175 | + | Single-stranded DNA-binding | Ralstonia |
| | | | | | | protein | phage GP4 |
| 12 | 7,994 | 8,218 | 225 | 74 | + | Predicted nucleic acid-binding | Ralstonia |
| | | | | | | protein | phage GP4 |
| 13 | 8,208 | 8,693 | 486 | 161 | + | Cation transport ATPase | Burkholderia |
| | | | | | | | phage BcepIL02 |
| 14 | 8,693 | 9,073 | 381 | 126 | + | Protein of unknown function | Ralstonia |
| | | | | | | | phage GP4 |
| 15 | 9,236 | 9,466 | 231 | 76 | + | Superfamily II DNA and RNA | Burkholderia |
| | | | | | | helicases | phage Bcep22 |
| 16 | 9,463 | 9,915 | 453 | 150 | + | NA | Ralstonia |
| | | | | | | | phage GP4 |
| 17 | 10,863 | 11,387 | 525 | 174 | + | Superfamily II DNA/RNA | Paraburkholderia |
| | | | | | | helicases, SNF2 family | thizoxinica |
| | | | | | | | HKI 454 |
| 18 | 11,374 | 12,030 | 857 | 218 | + | Cu2+-containing amine oxidase | Ralstonia |
| | | | | | | | phage GP4 |
| 19 | 12,088 | 12,525 | 438 | 145 | + | NA | Ralstonia |
| | | | | | | | phage GP4 |
| 20 | 12,522 | 12,821 | 300 | 99 | + | NA | Ralstonia |
| | | | | | | | solanacearum |
| 21 | 12,818 | 13,177 | 360 | 119 | + | N-acetylmuramoyl-L-alanine | Ralstonia |
| | | | | | | amidase | phage GP4 |
| 22 | 13,203 | 13,361 | 159 | 52 | + | NA | Ralstonia |
| | | | | | | | phage GP4 |
| 23 | 13,358 | 13,837 | 480 | 159 | + | FOG: TPR repeat | Ralstonia |
| | | | | | | | phage GP4 |
| 24 | 14,402 | 14,830 | 429 | 142 | − | FOG: TPR repeat, | Ralstonia |
| | | | | | | SEL 1 subfamily | phage GP4 |
| 25 | 15,188 | 16,826 | 639 | 212 | + | Phage terminase, small subunit | Burkholderia |
| | | | | | | | stagnalis |
| 26 | 16,080 | 16,283 | 204 | 67 | + | CTP synthase | Ralstonia |
| | | | | | | (UTP-ammonia lyase) | phage RSK1 |
| 27 | 16,323 | 16,631 | 309 | 102 | + | DNA segregation ATPase | Ralstonia |
| | | | | | | FtsK/SpoIIIE and related proteins | phage RSK1 |
| 28 | 16,699 | 16,857 | 159 | 52 | + | NA | Ralstonia |
| | | | | | | | phage GP4 |
| 29 | 16,854 | 16,997 | 144 | 47 | + | Mismatch repair ATPase | Ralstonia |
| | | | | | | (MutS family) | phage RSK1 |
| 30 | 16,994 | 17,344 | 351 | 116 | + | Dehydrogenases with | Ralstonia |
| | | | | | | different specificities | phage RSK1 |
| 31 | 17,356 | 17,577 | 222 | 73 | + | Predicted transcriptional | Ralstonia |
| | | | | | | regulator | phage GP4 |
| 32 | 17,558 | 18,319 | 762 | 253 | + | S-adenosylmethionine | Ralstonia |
| | | | | | | synthetase | phage RSK1 |
| 33 | 18,316 | 18,987 | 672 | 223 | + | Nucleoside phosphorylase | Ralstonia |
| | | | | | | | phage RSK1 |
| 34 | 19,008 | 19,712 | 705 | 234 | + | Uncharacterized protein | Ralstonia |
| | | | | | | conserved in bacteria | phage RSK1 |
| 35 | 19,709 | 19,912 | 204 | 67 | + | NA | Paraburkholderia |
| | | | | | | | fungorum |
| 36 | 20,219 | 20,434 | 216 | 71 | + | NA | Ralstonia |
| | | | | | | | phage RSK1 |
| 37 | 20,431 | 21,027 | 597 | 198 | + | Anthranilate | Ralstonia |
| | | | | | | phosphoribosyltransferase | phage GP4 |
| 38 | 21,024 | 21,224 | 201 | 66 | + | Methylase of polypeptide | Ralstonia |
| | | | | | | chain release factors | phage RSK1 |
| 39 | 21,200 | 21,451 | 252 | 83 | + | Uncharacterized | Ralstonia |
| | | | | | | conserved protein | phage GP4 |
| 40 | 21,549 | 21,740 | 192 | 63 | + | NA | Ralstonia |
| | | | | | | | phage GP4 |
| 41 | 22,825 | 24,147 | 1,323 | 440 | + | 17 | Ralstonia |
| | | | | | | | phage GP4 |
| 42 | 24,144 | 24,569 | 426 | 141 | + | Predicted phosphoglycerate | Ralstonia |
| | | | | | | mutase, AP superfamily | phage GP4 |
| 43 | 24,586 | 26,931 | 2,346 | 781 | + | P22_portal | Ralstonia |
| | | | | | | | phage GP4 |
| 44 | 27,006 | 27,296 | 291 | 96 | + | Predicted Fe—S oxidoreductases | Ralstonia |
| | | | | | | | phage GP4 |
| 45 | 27,433 | 27,882 | 450 | 149 | + | Signal transduction histidine | Ralstonia |
| | | | | | | kinase | phage GP4 |
| 46 | 27,892 | 28,341 | 450 | 149 | + | ABC-type uncharacterized | Burkholderia |
| | | | | | | transport system, periplasmic | ubonensis |
| | | | | | | component | |
| 47 | 28,351 | 28,548 | 198 | 65 | + | Hemolysins and related proteins | Ralstonia |
| | | | | | | containing CBS domains | phage GP4 |
| 48 | 28,624 | 28,911 | 288 | 95 | + | NADH dehydrogenase/ | Ralstonia |
| | | | | | | NADH:ubiquinone | phage GP4 |
| | | | | | | oxidoreductase 75 kD subunit | |
| | | | | | | (chain G) | |
| 49 | 29,062 | 30,171 | 1,110 | 369 | + | major capsid protein | Ralstonia |
| | | | | | | | phage GP4 |
| 50 | 30,249 | 30,722 | 474 | 157 | + | Histone acetyltransferase | Burkholderia |
| | | | | | | | phage BcepMigl |
| 51 | 30,779 | 31,318 | 540 | 179 | + | NA | Ralstonia |
| | | | | | | | phage GP4 |
| 52 | 31,322 | 31,984 | 663 | 220 | + | NA | Ralstonia |
| | | | | | | | phage GP4 |
| 53 | 31,981 | 32,601 | 621 | 206 | + | Cytochrome c, mono- and | Ralstonia |
| | | | | | | diheme variants | phage GP4 |
| 54 | 32,611 | 33,033 | 423 | 140 | + | NA | Ralstonia |
| | | | | | | | phage GP4 |
| 55 | 33,040 | 34,017 | 978 | 325 | + | PPE-repeat proteins | Ralstonia |
| | | | | | | | phage GP4 |
| 56 | 34,014 | 35,720 | 1,707 | 568 | + | Autotransporter adhesin | Ralstonia |
| | | | | | | | phage |
| | | | | | | | DU_RP_II |
| 57 | 35,720 | 36,001 | 282 | 93 | + | Pyruvate/2-oxoglutarate | Ralstonia |
| | | | | | | dehydrogenase complex, | phage GP4 |
| | | | | | | dihydrolipoamide | |
| | | | | | | acyltransferase (E2) | |
| | | | | | | component, and related enzymes | |
| 58 | 36,001 | 37,320 | 1,320 | 439 | + | Chaperone of endosialidase | Ralstonia |
| | | | | | | | phage GP4 |
| 59 | 37,406 | 38,278 | 873 | 290 | + | NA | Ralstonia |
| | | | | | | | phage GP4 |
| 60 | 38,290 | 40,023 | 1,734 | 577 | + | NA | Ralstonia |
| | | | | | | | phage GP4 |
| 61 | 40,023 | 40,343 | 321 | 106 | + | Phosphoserine aminotransferase | Burkholderia |
| | | | | | | | phage Bcep22 |
| 62 | 40,419 | 40,865 | 447 | 148 | + | Protein N-acetyltransferase | Ralstonia |
| | | | | | | | phage GP4 |
| 63 | 40,855 | 41,688 | 834 | 277 | + | NA | Ralstonia |
| | | | | | | | phage GP4 |
| 64 | 41,699 | 42,424 | 726 | 241 | + | 3′-phosphoadenosine | Ralstonia |
| | | | | | | 5′-phosphosulfate | phage GP4 |
| | | | | | | sulfotransferase (PAPS | |
| | | | | | | reductase)/FAD synthetase | |
| | | | | | | and related enzymes | |
| 65 | 42,421 | 43,950 | 1,530 | 509 | + | Predicted permeases | Ralstonia |
| | | | | | | | phage GP4 |
| 66 | 44,008 | 57,543 | 13,536 | 4,511 | + | DNA methylase | Burkholderia |
| | | | | | | | virus BcepIL02 |
| 67 | 58,023 | 58,244 | 222 | 73 | − | Thiamine biosynthesis | Ralstonia |
| | | | | | | enzyme ThiH and related | phage GP4 |
| | | | | | | uncharacterized enzymes | |
| 68 | 58,287 | 58,598 | 312 | 103 | − | Predicted metal-binding, possibly | Ralstonia |
| | | | | | | nucleic acid-binding protein | phage GP4 |
| 69 | 58,762 | 59,355 | 594 | 197 | + | NA | Ralstonia |
| | | | | | | | sp. GV074 |
| 70 | 59,321 | 59,968 | 648 | 216 | − | Predicted SAM-dependent | Cronobacter |
| | | | | | | methyltransferases | phage PBES 02 |
| 71 | 60,035 | 60,262 | 228 | 75 | + | Spermidine/putrescine-binding | Burkholderia |
| | | | | | | periplasmic protein | phage KL19 |
| 72 | 60,355 | 60,735 | 381 | 126 | + | Seryl-RNA synthetase | Ralstonia |
| | | | | | | | phage GP |
| 73 | 60,695 | 61,114 | 420 | 139 | + | putative uroporphyrinogen-III | Pseudomonas |
| | | | | | | C-methyltransferase | amygdali |
| 74 | 61,114 | 61,647 | 534 | 177 | + | Bacteriophage Rz lysis protein | Ralstonia |
| | | | | | | | phage GP4 |
| 75 | 61,352 | 61,597 | 246 | 81 | + | lipoprotein LpqB; Provisional | Burkholderia |
| | | | | | | | phage DC1 |

| | | | | homolog |
|---|---|---|---|---|
| ORF | product | E-value | Ident. | Accession no. |
| 1 | hypothetical | 2.00E−39 | 100% | AXG67755.1 |
| | protein | | | |
| 2 | tyrosine | 0 | 93% | AXG67754.1 |
| | recombinase | | | |
| 3 | Pyocin | 5.00E−39 | 72% | WP_060640377.1 |
| | activator | | | |
| | protein PrtN | | | |
| 4 | hypothetical | 3.00E−177 | 73% | AXG67751.1 |
| | protein | | | |
| 5 | DNA-binding | 6.00E−37 | 83% | AXG67748.1 |
| | protein | | | |
| 6 | RecT-like protein | 0 | 94% | AXG67747.1 |
| 7 | RecB-like | 0 | 95% | AXG67746.1 |
| | nuclease | | | |
| 8 | Fis family | 1.00E−61 | 6.00E−01 | AXG67745.1 |
| | transcriptional | | | |
| | regulator | | | |
| 9 | Transcriptional | 9.00E−102 | 78% | AXG67744.1 |
| | regulator | | | |
| 10 | transcriptional | 6.00E−71 | 93% | AXG67743.1 |
| | regulator | | | |
| tRNA | tRNA-Ser | 3.00E−22 | 90% | AXG67742.1 |
| 11 | single-stranded | 2.00E−80 | 75% | AXG67740.1 |
| | DNA-binding | | | |
| | protein | | | |
| 12 | hypothetical | 2.00E−36 | 81% | AXG67738.1 |
| | protein | | | |
| 13 | hypothetical | 2.00E−58 | 60% | YP_002922687.1 |
| | protein | | | |
| | BcepIL02_gp15 | | | |
| 14 | DUF 1364 | 5.00E−64 | 79% | AXG67736.1 |
| | family protein | | | |
| 15 | hypothetical | 2.00E−31 | 74% | NP_944252.1 |
| | protein | | | |
| 16 | hypothetical | 2.00E−71 | 100% | AXG67732.1 |
| | protein | | | |
| 17 | replication | 4e−50 | 62% | CBW74437.1 |
| | protein 15 | | | |
| 18 | hypothetical | 7.00E−151 | 100% | AXG67729.1 |
| | protein | | | |
| 19 | hypothetical | 2.00E−49 | 61% | AXG67728.1 |
| | protein | | | |
| 20 | hypothetical | 1.00E−27 | 56% | WP_019717901.1 |
| | protein | | | |
| 21 | hypothetical | 1.00E−38 | 58% | AXG67726.1 |
| | protein | | | |
| 22 | hypothetical | 4.00E−07 | 83% | AXG67725.1 |
| | protein | | | |
| 23 | IQ | 2.00E−51 | 59% | AXG67724.1 |
| | calmodulin-binding | | | |
| | domain-containing | | | |
| | family protein | | | |
| 24 | hypothetical | 7.00E−86 | 94% | AXG67722.1 |
| | protein | | | |
| 25 | terminase small | 9.00E−64 | 65% | WP_059906112.1 |
| | subunit | | | |
| 26 | hypothetical | 1.00E−41 | 100% | YP_008853825.1 |
| | protein | | | |
| 27 | hypothetical | 2.00E−67 | 100% | YP_008853824.1 |
| | protein | | | |
| 28 | hypothetical | 3.00E−26 | 96% | AXG67717.1 |
| | protein | | | |
| 29 | hypothetical | 9.00E−23 | 94% | YP_008853823.1 |
| | protein | | | |
| 30 | hypothetical | 1.00E−67 | 88% | YP_008853822.1 |
| | protein | | | |
| 31 | transcriptional | 7.00E−41 | 93% | AXG67713.1 |
| | regulator | | | |
| 32 | hypothetical | 1.00E−43 | 44% | YP_008853821.1 |
| | protein | | | |
| 33 | hypothetical | 3.00E−137 | 99% | YP_008853820.1 |
| | protein | | | |
| 34 | hypothetical | 8.00E−45 | 80% | YP_008853814.1 |
| | protein | | | |
| 35 | hypothetical | 4.00E−12 | 50% | WP_074769973.1 |
| | protein | | | |
| 36 | hypothetical | 5.00E−43 | 100% | YP_008853815.1 |
| | protein | | | |
| 37 | hypothetical | 4.00E−30 | 93% | AXG67709.1 |
| | protein | | | |
| 38 | hypothetical | 0.037 | 94% | YP_008853811.1 |
| | protein | | | |
| 39 | hypothetical | 2.00E−43 | 96% | AXG67707.1 |
| | protein | | | |
| 40 | CoA ester lyase | 3.00E−38 | 100% | AXG67706.1 |
| 41 | terminase large | 0.00E+00 | 98% | AXG67705.1 |
| | subunit | | | |
| 42 | hypothetical | 5E−99 | 96% | AXG67704.1 |
| | protein | | | |
| 43 | portal protein | 0 | 99% | AXG67702.1 |
| 44 | hypothetical | 0.0001 | 100% | AXG67701.1 |
| | protein | | | |
| 45 | hypothetical | 8.00E−102 | 98% | AXG67701.1 |
| | protein | | | |
| 46 | hypothetical | 7.00E−30 | 78% | WP_060969604.1 |
| | protein | | | |
| 47 | CsrA-like | 4.00E−37 | 98% | AXG67699.1 |
| | transcriptional | | | |
| | regulator | | | |
| 48 | hypothetical | 3.00E−61 | 98% | AXG67698.1 |
| | protein | | | |
| 49 | Major capsid | 0.00E+00 | 100% | AXG67697.1 |
| | protein | | | |
| 50 | Virion associated | 3.00E−44 | 53% | WP_090835605.1 |
| | protein | | | |
| 51 | hypothetical | 9.00E−72 | 96% | AXG67778.1 |
| | protein | | | |
| 52 | Virion associated | 2.00E−156 | 98% | AXG67777.1 |
| | protein | | | |
| 53 | hypothetical | 6.00E−135 | 89% | AXG67776.1 |
| | protein | | | |
| 54 | Virion-associated | 2.00E−94 | 99% | AXG67775.1 |
| | phage protein | | | |
| 55 | virion-associated | 0.00E+00 | 99% | AXG67774.1 |
| | phage protein | | | |
| 56 | minor tail protein | 7.00E−83 | 40% | AXG67773.1 |
| 57 | Virion-associated | 5.00E−60 | 99% | AXG67772.1 |
| | phage protein | | | |
| 58 | putative tail | 0.00E+00 | 99% | AXG67771.1 |
| | fiber protein | | | |
| 59 | tail fiber protein | 2.00E−165 | 85% | AXG67770.1 |
| 60 | Virion-associated | 0 | 99% | AXG67769.1 |
| | phage protein | | | |
| 61 | virion-associated | 9.00E−06 | 41% | AXG67768.1 |
| | phage protein | | | |
| 62 | Acyl-CoA | 2.00E−106 | 100% | AXG67767.1 |
| | N-acyltransferase | | | |
| 63 | Virion-associated | 0.00E+00 | 100% | AXG67766.1 |
| | phage protein | | | |
| 64 | PAPS reductase- | 3.00E−180 | 99% | AXG67765.1 |
| | like protein | | | |
| 65 | virion-associated | 0.00E+00 | 9.90E−01 | AXG67764.1 |
| | phage protein | | | |
| 66 | lytic | 0.00E+00 | 68% | YP_002922742.1 |
| | transglycosylase | | | |
| | domain-containing | | | |
| | DarB protein | | | |
| 67 | hypothetical | 8.00E−17 | 57% | AXG67762.1 |
| | protein | | | |
| 68 | hypothetical | 1.00E−67 | 97% | AXG67761.1 |
| | protein | | | |
| 69 | DUF3380 domain- | 1.00E−72 | 61% | ″AXG67760.1 |
| | containing N- | | | |
| | acetylmuramidase | | | |
| | (lysozyme-like) | | | |
| | family protein | | | |
| | (endolysin) | | | |
| 70 | hypothetical | 8.00E−08 | 37% | WP_058379124.1 |
| | protein | | | |
| | ADU18_0048 | | | |
| 71 | hypothetical | 3.00E−03 | 35% | YP_009188912.1 |
| | protein | | | |
| 72 | chemotaxis | 1.00E−85 | 100% | AXG67759.1 |
| | protein | | | |
| 73 | lipoprotein | 2.00E−31 | 31% | AXG67758.1 |
| 74 | Rz-like phage | 1.00E−98 | 93% | AXG67757.1 |
| | lysis protein | | | |
| 75 | Rz1 | 6.E−04 | 57% | AXG67756.1 |

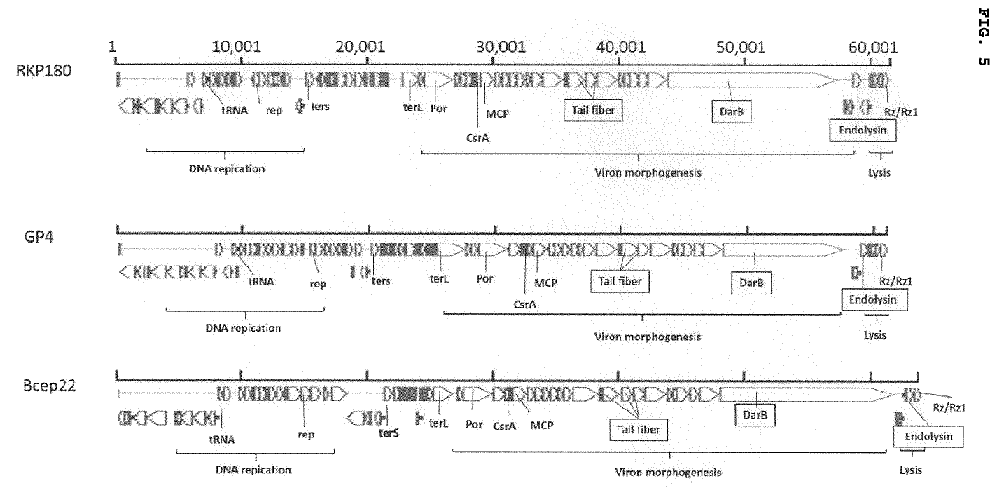

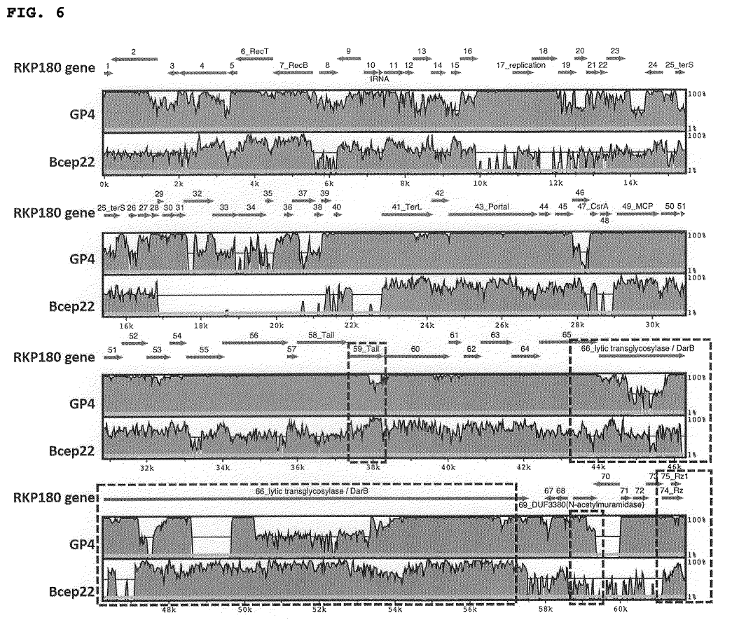

Maps of major genes of the genomes were drawn using MiGAP, and RKP180 and known Ralstonia phage strain GP4 and Burkholderia cepacia phage strain Bcep22 were compared (FIG. 5). Moreover, the entire base sequences were also compared using comparative genomic software LAGAN (FIG. 6).

The genomic structure was similar to those of Ralstonia phage strain GP4 and Burkholderia cepacia phage strain Bcep22 (FIGS. 5 and 6).

RKP180 genome is characterized by having DarB gene, which is a lysozyme fused restriction mechanism defense gene, having genes encoding three lytic enzymes of DarB, an endolysin and Rz/Rz1, having a gene encoding biofilm formation-inhibiting protein, CsrA, and having genes encoding two different tail fiber proteins.

Furthermore, RKP180 genome is characterized in that, while the genomic structure of RKP180 is very similar to that of GP4, the proteins encoded by DarB gene (ORF66), two lytic enzyme genes (ORF69 and ORF75) and one tail fiber gene (ORF59) have low identities to the corresponding homologs of GP4 (FIGS. 6 to 8).

ORF66 of RKP180 encodes a fusion protein in which a lysozyme-like lytic enzyme and DarB [defence against restriction] are fused (FIG. 7). Those which were homologous to the fusion protein were DarB-like antirestriction protein (coverage of 90% and identity of 68%) of Burkholderia virus BcepILO2, soluble lytic transglycosylase/helicase/methylase protein (coverage of 89% and identity of 68) of Burkholderia virus DC1 and DarB-like antirestriction protein (coverage of 97% and identity of 67%) of Burkholderia virus Bcep22 (Table 3).

On the other hand, the identity to DarB-like antirestriction protein of the closest related species, Ralstonia phage strain GP4, was low with a coverage of 53% and an identity of 93% (Table 3). This was because the methylase domain and the helicase domain in the DarB domain, which is for defense against the restriction mechanism of the host bacterium, are incomplete although the lysozyme-like domain is conserved and because the homologous region is limited (Table 4, FIGS. 6 and 7 and Document 1 (Gill J J, Summer E J, Russell W K, Cologna S M, Carlile T M, Fuller A C, Kitsopoulos K, Mebane L M, Parkinson B N, Sullivan D, Carmody L A, Gonzalez C F, LiPuma J J, Young R. 2011. Genomes and characterization of phages Bcep22 and BcepIL02, founders of a novel phage type in Burkholderia cenocepacia. J Bacteriol 193:5300-5313)).

TABLE 3

| Top | Protein | Species | Coverage | Identity | Accession |
|---|---|---|---|---|---|
| 1 | DarB-like antirestriction | Burkholderia virus | 90% | 68% | YP_002922742.1 |
| | protein | BcepIL02 | | | |
| 2 | multidomain (soluble lytic | Burkholderia virus | 89% | 68% | YP_006589997.1 |
| | transglycosylase/helicase/ | DC1 | | | |
| | methylase) protein | | | | |
| 3 | DarB-like antirestriction | Burkholderia virus | 97% | 67% | NP_944303.1 |
| | protein | Bcep22 | | | |
| 4 | DarB-like antirestriction | Burkholderia virus | 77% | 65% | YP_007236815.1 |
| | protein | Bcepmigl | | | |
| 5 | PLxRFG domain- | Caballeronia | 50% | 68% | WP_051996747.1 |
| | containing protein | zhejiangensis | | | |
| 6 | DarB-like antirestriction | Ralstonia phage GP4 | 53% | 93% | AXG67763.1 |
| | protein | | | | |

TABLE 4

| RKP180 | Domain | | | | | | |
|---|---|---|---|---|---|---|---|
| Position | | Motif/ | | Comparison with GP4 | | Comparison with Bcep22 | |
| (aa) | Domain | Accession | Position | Coverage | Identity | Coverage | Identity |
| 1-4511 | Whole Region | | 1-4511 | 53% | 93% | 97% | 67% |
| 1-200 | lytic transglycosylase | LT-like/ | 64-178 | 88% | 85% | 66% | 70% |
| | (lysozyme-like) | cd00254 | | | | | |
| 1500-1800 | methylase | YtxK/ | 1546-1800 | Not Detected | | 97% | 75% |
| | | COG0827 | | | | | |
| 1801-2400 | | COG4646/ | 1820-2353 | 96% | 41% | 100% | 64% |
| | | COG4646 | | | | | |

| | | | | | | |
|---|---|---|---|---|---|---|
| 2300-2900 | helicase | HepA/ | 2296-2878 | Not Detected | 100% | 81% |
| | | COG0553 | | | | |

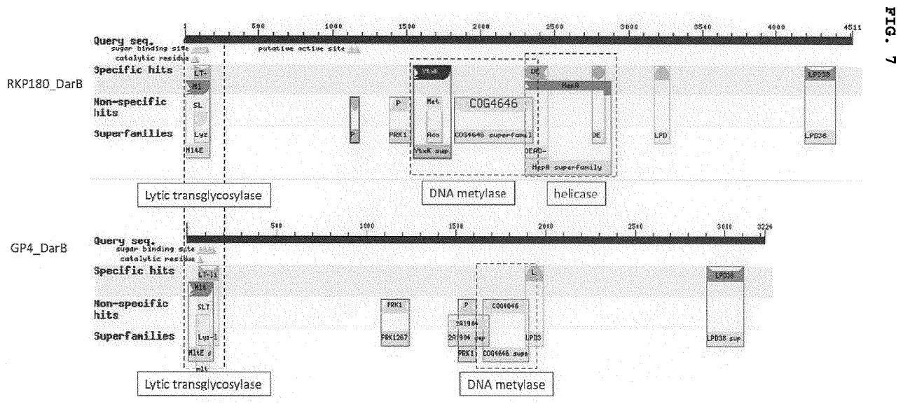

ORF69 of RKP180 encoded an endolysin having N-acetylmuramidase activity [DUF3380 domain-containing protein], which is a lytic enzyme (Document 2 (Rodriguez-Rubio L, Gerstmans H, Thorpe S, Mesnage S, Lavigne R, Briers Y. 2016. DUF3380 Domain from a Salmonella Phage Endolysin Shows Potent N-Acetylmuramidase Activity. Appl Environ Microbiol 82:4975-4981) and Document 3 (Love M J, Bhandari D, Dobson R C J, Billington C. 2018. Potential for Bacteriophage Endolysins to Supplement or Replace Antibiotics in Food Production and Clinical Care. Antibiotics (Basel) 7:17)). As a result of the homology search with BLASTP, the identity to ORF65 (AXG67760.1) of strain GP4 was the highest (coverage of 100% and identity of 82%, Table 5). The identity of the N-terminus (1-100 aa) region was high, namely 96, (coverage of 100%), while the identity of the C-terminus (101-197 aa) was 68% (coverage of 100%), which was lower than that of the N-terminus (Table 6 and FIG. 8).

TABLE 5

| Top | Protein | Species | Coverage | Identity | Accession |
|---|---|---|---|---|---|
| 1 | DUF3380 domain-containing protein | Ralstonia phage GP4 | 100% | 82% | AXG67760.1 |
| 2 | DUF3380 domain-containing protein | Cupriavidus oxalaticus | 98% | 64% | WP_063238227.1 |
| 3 | conserved hypothetical protein | Cupriavidus necator | 98% | 63% | SCU94272.1 |
| 4 | DUF3380 domain-containing protein | Ralstonia sp. UNC404CL21Col | 96% | 63% | WP_027681351.1 |
| 5 | DUF3380 domain-containing protein | Cupriavidus taiwanensis | 98% | 60% | WP_115662684.1 |
| 6 | N-acetylmuramidase family protein | Pandoraea norimbergensis | 97% | 59% | WP_058379124.1 |

TABLE 6

| | RKP180 | GP4 | | |
|---|---|---|---|---|
| | Position | Position | | |
| | (aa) | (aa) | Coverage | Identity |
| Whole Region | 1-197 | 1-202 | 100% | 82% |
| N-Terminus | 1-100 | 1-100 | 100% | 96% |
| C-Terminus | 101-197 | 101-202 | 100% | 68% |

In RKP180, spanin lytic enzymes of Rz and Rz1 were encoded by ORF74 and ORF75, respectively. Rz1 is in Rz gene with a different reading frame, and many phages have both Rz and Rz1 (Document 1).

GP4, which is the most closely related strain of RKP180, has Rz, and the identity of ORF74 of RKP180 and the Rz-like lysis protein of GP4 was 93% (coverage of 100%), which was a high identity (Table 7). GP4, however, does not have Rz1 (Document 4 (Wang R, Cong Y, Mi Z, Fan H, Shi T, Liu H, Tong Y. 2019. Characterization and complete genome sequence analysis of phage GP4, a novel lytic Bcep22-like podovirus. Arch Virol 164:2339-2343)). On the other hand, although related strains, Burkholderia virus DC1 and Burkholderia virus Bcep22, have homologs of both Rz and Rz1, the identities to ORF74 (Rz) and ORF75 (Rz1) of RKP180 were 49% (coverage of 85% and 57% (coverage of 86%), respectively in DC1 and 46% (coverage of 100%) and 56% (coverage of 86%), respectively in Bcep22, which were all low identities (Tables 7 and 8).

TABLE 7

| Top | Protein | Species | Coverage | Identity | Accession |
|---|---|---|---|---|---|
| 1 | Rz-like lysis protein | Ralstonia phage GP4 | 100% | 93% | AXG67756.1 |
| 2 | Rz-like phage lysis protein | Burkholderia virus Bcep22 | 100% | 46% | NP_944308.1 |
| 3 | Rz | Burkholderia virus DC1 | 85% | 49% | YP_006590002.1 |
| 4 | hypothetical protein | Paraburkholderia hospita | 87% | 42% | WP_090835650.1 |
| 5 | lysis protein | Pseudomonas viridiflava | 60% | 44% | WP_122665472.1 |
| 6 | hypothetical protein | Pseudomonas fluorescens | 96% | 35% | VVO19773.1 |
| | PS712_04136 | | | | |

TABLE 8

| Top | Protein | Species | Coverage | Identity | Accession |
|---|---|---|---|---|---|
| 1 | hypothetical protein | Pseudomonas phage Epa38 | 98% | 43% | QIQ65891.1 |
| | 38_00056 | | | | |
| 2 | Formylglycine- | Pseudomonas syringae | 72% | 52% | GBH14702.1 |
| | generating enzyme | pv. actinidiae | | | |
| 3 | hypothetical protein | Pseudomonas phage | 98% | 42% | ASZ72374.1 |
| | vBPaeSS218_00076 | vB_PaeS_S218 | | | |
| 4 | hypothetical protein | Pseudomonas brassicacearum | 70% | 47% | ROM94378.1 |
| | BK658_17630 | | | | |
| 5 | hypothetical protein | Pseudomonas sp. 31 R17 | 92% | 44% | CRM53430.1 |
| 6 | hypothetical protein | Paraburkholderia tuberum | 85% | 44% | SDR18245.1 |
| | SAMN05445850_3160 | | | | |
| 7 | hypothetical protein | Pseudomonas simiae | 76% | 44% | AIB35092.1 |
| | PS417_05805 | | | | |
| 8 | Rz1 | Burkholderia virus DC1 | 86% | 57% | YP_006590003.1 |
| 9 | hypothetical protein | Pseudomonas chlororaphis | 65% | 48% | AZC35926.1 |
| | C4K37_1524 | subsp. piscium | | | |
| 10 | hypothetical protein | Pseudomonas sp. OV546 | 79% | 47% | SFU95081.1 |
| | SAMN05428951_10733 | | | | |
| 11 | Rz1-like phage lysis | Burkholderia virus Bcep22 | 86% | 56% | YP_009173772.1 |
| | lipoprotein | | | | |

It is believed that RKP180 lyses the host bacterium efficiently due to the functions of the those three lytic enzymes and that the infection cycle is thus efficient. It is also believed that, due to the differences in the lytic enzymes, RKP180 has an infection cycle having different characteristics from those of GP4, which is the most closely related strain (see below, Document 4).

It was found that ORF47 of RKP180 encodes CsrA [carbon storage regulator] protein (Table 9). CsrA is a gene which suppresses biofilm formation (Document 5 (Lynch K H, Stothard P, Dennis J J. 2012. Characterization of DC1, a Broad-Host-Range Bcep22-Like Podovirus. Appl Environ Microbiol 78:889-891)). In Escherichia coli, the expression of CsrA suppresses biofilm formation and inhibits the development of biofilm by increasing motility (Document 6 (Lu T K, Collins J J. 2009. Engineered bacteriophage targeting gene networks as adjuvants for antibiotic therapy. Proc Natl Acad Sci USA 106:4629-4634) and Document 7 (Jackson D W, Suzuki K, Oakford L, Simecka J W, Hart M E, Romeo T. 2002. Biofilm Formation and Dispersal under the Influence of the Global Regulator CsrA of Escherichia coli. J Bacteriol 184:290-301)). Furthermore, in related Burkholderia Bcep22 group phages, relevance to the inhibition of biofilm formation is suggested (Document 5). Moreover, bacterial wilt disease is caused by the biofilm formation by Ralstonia solanacearum, and Ralstonia solanacearum has genes involved in the biofilm formation and the motility, which are known to be regulated by CsrA (Document 8 (Clough S J, Lee K E, Schell M A, Denny T P. 1997. A two-component system in Ralstonia (Pseudomonas) solanacearum modulates production of PhcA-regulated virulence factors in response to 3-hydroxypalmitic acid methyl ester. J Bacteriol 179:3639-3648), Document 9 (Brown D G, Allen C. 2004. Ralstonia solanacearum genes induced during growth in tomato: an inside view of bacterial wilt. Molecular Microbiology 53:1641-1660) and Document 10 (Hikichi Y, Mori Y, Ishikawa S, Hayashi K, Ohnishi K, Kiba A, Kai K. 2017. Regulation Involved in Colonization of Intercellular Spaces of Host Plants in Ralstonia solanacearum. Front Plant Sci 8:967)).

From the above points, RKP180 having CsrA inhibits the biofilm formation by Ralstonia solanacearum and lyses Ralstonia solanacearum, and as a result, a high control effect of RKP180 is expected. Although only GP4, of Ralstonia phages, has homologous genes, the only Ralstonia phage in which the control effect was confirmed is RKP180.

TABLE 9

| Top | Protein | Species | Coverage | Identity | Accession |
|---|---|---|---|---|---|
| 1 | carbon storage regulator | Ralstonia phage GP4 | 100% | 98% | AXG67699.1 |
| 2 | carbon storage regulator | Paraburkholderia zhejiangensis | 93% | 67% | WP_034473805.1 |
| 3 | carbon storage regulator | Burkholderia sp. BDU8 | 90% | 66% | WP_066491652.1 |
| 4 | carbon storage regulator | Paraburkholderia hospita | 93% | 61% | WP_090835601.1 |
| 5 | carbon storage regulator | Burkholderia virus DC1 | 95% | 60% | YP_006589976.1 |

ORF58 and ORF59 of RKP180 encoded two different tail fiber proteins (Table 10 and Table 11). The tail fiber proteins specifically bind to receptor molecules on the surface layer of the host bacterium, and this specificity determines the host specificity, namely the host range, of the bacteriophage (Documents 1 and 11 (Bartual S G, Otero J M, Garcia-Doval C, Llamas-Saiz A L, Kahn R, Fox G C, van Raaij M J. 2010. Structure of the bacteriophage T4 long tail fiber receptor-binding tip. Proc Natl Acad Sci USA 107:20287-20292)).

ORF58 of RKP180 had the highest identity to the tail fiber protein of ORF76 (AXG67771.1) of Ralstonia phage GP4 (coverage of 100% and identity of 99%) (Table 10).

TABLE 10

| Top | Protein | Species | Coverage | Identity | Accession |
|---|---|---|---|---|---|
| 1 | putative tail fiber protein | Ralstonia phage GP4 | 100% | 99% | AXG67771.1 |
| 2 | hypothetical protein | Ralstonia solanacearum | 50% | 54% | WP_049842194.1 |
| 3 | hypothetical protein | Ralstonia solanacearum | 50% | 53% | WP_064049197.1 |
| 4 | putative phage tail fiber | Ralstonia phage RSJ5 | 72% | 36% | YP_009218128.1 |
| | protein | | | | |
| 5 | tail fiber domain-containing | Burkholderia sp. b14 | 30% | 53% | WP_076795202.1 |
| | protein | | | | |

Moreover, ORF59 of RKP180 had the highest identity to ORF75 (AXG67770.1) of Ralstonia phage GP4 (coverage of 100% and identity of 85%) (Table 11). When the domain was searched using InterPro 78.1, the region of the 109th to the 280th amino acids at the C-terminus of ORF59 was identified as the receptor-binding domain of the short tail fibre protein gp12 SUPERFAMILY (SSF88874). This domain is the host recognition site of short tail fiber gp12 of T4 phage (Documents 1 and 12 (Leiman P G, Arisaka F, Raaij M L, Kostyuchenko V A, Aksyuk A A, Kanamaru S, Rossmann M G. 2010. Morphogenesis of the T4 tail and tail fibers. Virology J 7:355). When homology search with BLASTP was conducted using the region of the 109th to the 280th amino acids corresponding to the host recognition site domain of ORF59 in RKP180, the identity to the homologous site of the tail fiber protein of Burkholderia virus DC1 was the highest (coverage of 100% and identity of 85%), and the identity to Ralstonia phage GP4 was low, namely 77% (coverage of 100%, Table 12). Thus it is believed from the results that the difference in the sequence of the host recognition site of the ORF59 homolog causes the difference in the host ranges of Ralstonia phages RKP180 and GP4.

TABLE 11

| Top | Protein | Species | Coverage | Identity | Accession |
|---|---|---|---|---|---|
| 1 | tail fiber protein | Ralstonia phage GP4 | 100% | 85% | AXG67770.1 |
| 2 | phage tail fiber protein | Burkholderia virus Bcep22 | 98% | 70% | NP_944294.2 |
| 3 | tail fiber protein | Burkholderia virus DC1 | 98% | 70% | YP_006589989.1 |
| 4 | Tail fiber | Burkholderia virus Bcepmigl | 98% | 69% | YP_007236807.1 |
| 5 | putative tail fiber protein | Burkholderia virus BcepIL02 | 98% | 69% | YP_002922733.1 |

TABLE 12

| Top | Protein | Species | Coverage | Identity | Accession |
|---|---|---|---|---|---|
| 1 | tail fiber protein | Burkholderia virus DC1 | 100% | 85% | YP_006589989.1 |
| 2 | phage tail fiber protein | Burkholderia virus Bcep22 | 100% | 85% | NP_944294.2 |
| 3 | Tail fiber | Burkholderia virus Bcepmigl | 100% | 85% | YP_007236807.1 |
| 4 | putative tail fiber protein | Burkholderia virus BcepIL02 | 100% | 84% | YP_002922733.1 |
| 5 | tail fiber protein | Ralstonia phage GP4 | 100% | 77% | AXG67770.1 |

### (5) Summary

From the above results, it was found that bacteriophage RKP180 has the following properties.
The genome of RKP180 is double-stranded DNA and has a circular permuted [CP] structure with a terminal redundancy [TR] sequence.
Belongs to the family of podovirus of the order Caudovirales.
The genome size is 61,696 bp.
The GC content is 64.58%.
76 genes (75 open reading frames (ORFs) and one tRNA) are included.
Similar to known bacteriophages (the genus Bcep22 virus of the family of podovirus) infecting Burkholderia cenocepacia.
The most closely related strain was known Ralstonia phage strain GP4 (coverage of 77% and identity of 97%).
Has restriction mechanism defense gene DarB.
Has genes encoding three lytic enzymes of DarB, an endolysin and Rz/Rz1.
Has the biofilm formation-inhibiting CsrA gene.
Has genes encoding two different tail fiber proteins.

### Example 3

Separation of Ralstonia solanacearum:
(1) Separation from Disease Plant

A stem of a disease plant was cut, and bacterial ooze was collected. The bacterial ooze was appropriately diluted with distilled water and applied to Modified SMSA medium, and formed colonies was separated onto CPG agar plates (10 g of peptone, 1 g of casamino acid, 5 g of glucose and 17 g of agar per 1 L).

(2) Separation from Soil

Soil and water were mixed well and left still, and then the supernatant was separated. The separated supernatant was appropriately diluted with water, and the diluted solution was applied to Modified SMSA medium. Formed colonies were separated on CPG agar plates.

<Modified SMSA Medium (per 1 L)>

| | | |
|---|---|---|
| Peptone | 10 | g |
| Glucose | 5 | g |
| Casamino acid | 1 | g |
| Agar | 17 | g |
| Bacitracin (10 mg/mL) | 2.5 | mL |
| Polymyxin B sulfate (50 mg/mL) | 2 | mL |
| Chloramphenicol (10 mg/mL) | 0.5 | mL |
| Penicillin G potassium (1 mg/mL) | 0.5 | mL |
| Crystal violet (1 mg/mL) | 5 | mL |
| Tetrazolium chloride (10 mg/mL) | 5 | mL |

### (3) Results

One strain of Ralstonia solanacearum was separated from one field by the above method, and 70 Ralstonia solanacearum strains or more in total from the hosts of the family Solanaceae, the family Zingiberaceae, the family Lamiaceae and the like were separated.

### Example 4

### Examination of Host Range of RKP180:

### (1) Presence or Absence of Infection

The presence or the absence of infection was examined by a plaque assay or a spot test.

### (2) Plaque Assay

The Ralstonia solanacearum strains were cultured at 28° C. overnight in CPG medium (10 g of peptone, 1 g of casamino acid and 5 g of glucose per 1 L). The bacterial culture solutions were adjusted to OD600 of 0.25 with CPG medium. A serial dilution of a phage solution was prepared and mixed with the bacterial culture solutions. After leaving still at 28° C. for 30 minutes, 3 ml of top agar (3 g of peptone, 0.3 g of casamino acid, 1.7 g of glucose and 5 g of agar per 1 L) and the bacterium/phage mixtures were mixed and layered onto CPG agar plates. After culturing at 28° C. overnight, the presence or the absence of infection was observed with the presence or the absence of plaques.

### (3) Spot Test

The Ralstonia solanacearum strains were cultured at 28° C. overnight in CPG medium. The bacterial culture solutions were adjusted to OD600 of 0.25 with CPG medium. The bacterial solutions in a volume of 250 μL and 3 ml of top agar were mixed and layered onto CPG agar plates. After the top agar was hardened, a phage solution was spotted, and the presence or the absence of plaques was observed to examine the presence or the absence of infection.

### (4) Results

It was found that RKP180 infects all the 100 Ralstonia solanacearum strains or more in total from the hosts of the family Solanaceae, the family Zingiberaceae, the family Lamiaceae and the like separated from the nature, including the 34 strains listed in Table 13, which can be obtained from Genebank Project, NARO, National Agriculture and Food Research Organization (Tsukuba, Ibaraki).

TABLE 13

| Bacterial Strain | Source of Separation |
|---|---|
| MAFF107624 | Tomato |
| MAFF211266 | Tomato |
| MAFF211270 | Tomato |
| MAFF211543 | Tomato |
| MAFF301859 | Tomato |
| MAFF311644 | Tomato |
| MAFF730103 | Tomato |
| MAFF730131 | Tomato |
| MAFF302745 | Tomato |
| MAFF311632 | Tomato |
| MAFF211536 | Tomato |
| MAFF331041 | Tomato |
| MAFF730139 | Eggplant |
| MAFF211280 | Eggplant |
| MAFF211533 | Eggplant |
| MAFF211468 | Chili pepper |
| MAFF211516 | Bell pepper and sweet green pepper |
| MAFF311101 | Bell pepper and sweet green pepper |
| MAFF311102 | Bell pepper and sweet green pepper |
| MAFF211479 | Ginger |
| MAFF211471 | Ginger |
| MAFF211483 | Ginger |
| MAFF211484 | Ginger |
| MAFF211486 | Ginger |
| MAFF211272 | Curcuma |
| MAFF211276 | Curcuma |
| MAFF211278 | Curcuma |
| MAFF211490 | Myoga ginger |
| MAFF211492 | Myoga ginger |
| MAFF211497 | Myoga ginger |
| MAFF211476 | Ginger |
| MAFF211414 | Potato |
| MAFF211429 | Potato |
| MAFF301558 | Potato |

### Example 5

### Evaluation of Infection Cycle of RKP180 by One-Step Growth Method

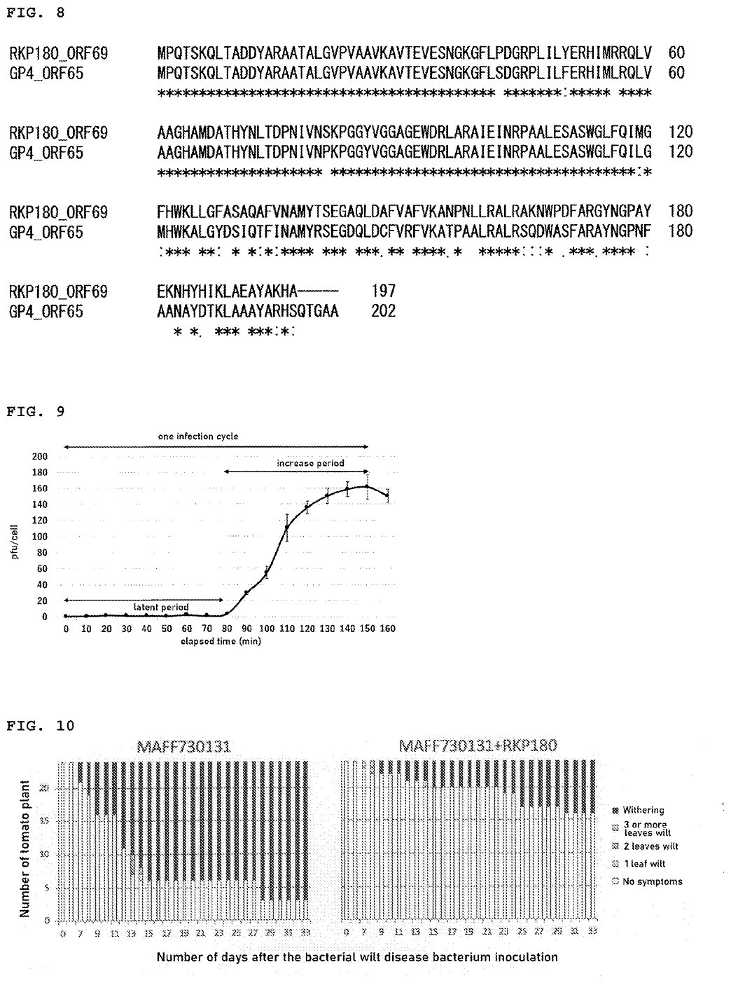

After mixing 990 μL of a culture solution of Ralstonia solanacearum strain MAFF730103 cultured to OD660 of 0.13 in CPG medium and 10 μL of a RKP180 phage solution (7×10⁸ pfu/mL), the mixture was left still at room temperature to adsorb the bacterium and the phage. After 10 minutes, the mixture was centrifuged at 5,000×g for 10 minutes, and the supernatant was collected. Then, the plaque assay was conducted, and the number of adsorbed phages was counted. The precipitates were re-suspended in 1 mL of CPG medium, and 50 μL was added to 24,950 μL of CPG medium, followed by culturing with shaking at 28° C. For 160 minutes after starting shaking, a 10-μL sample was taken every 10 minutes and mixed with 990 μL of the CPG medium to prevent re-adsorption. The mixtures or diluted solutions which were diluted in a stepwise manner according to the need in a volume of 100 μL and 250 μL of a culture solution of strain MAFF730103 which was adjusted to OD660 of 0.22 to 0.24 were mixed, and the total volumes thereof were added immediately to top agar, mixed and then layered onto CPG medium. The numbers of formed plaques were counted, and potency was determined. From the potency and the adsorbed phage numbers at the each time point, the phage number generated from one host bacterium cell (burst size) was calculated. The results are shown in FIG. 9 and summarized in Table 14.

TABLE 14

| Latent Period | Rise Period | One Infection Cycle | Burst Size |
|---|---|---|---|
| 80 minutes | 70 minutes | 150 minutes | 162 ± 8 pfu |

From the above results, it was found that the latent period of RKP180 is 80 minutes and that one infection cycle is 150 minutes. This is a shorter infection cycle than that of GP4, which is the most closely related species and is a Ralstonia phage, and the burst size was large, namely 162±8 pfu (Document 5). As the results of the genomic analysis, the efficient infection cycle is thought to be achieved because the bacteriophage has restriction mechanism defense gene DarB and the genes encoding the three lytic enzymes (the DarB, an endolysin and Rz/Rz1).

### Example 6

### Examination of Control Effect of RKP180

Seedlings (24 seedlings per test zone) of a large fruit variety, Sekaiichi, in a cell seedling tray were immersed in a 3×10⁹ pfu/mL phage solution for two minutes. The control zone was not treated. On the next day, the seedlings were planted in pots, and 5 mL of a bacterial solution of Ralstonia solanacearum strain MAFF730131 which was adjusted to OD660=0.1 (corresponding to approximate 1×10⁸ cfu/mL) was poured to the roots of the seedlings, followed by observation for 33 days (FIG. 10).

On the 33rd day after the bacterium inoculation, 21 of the 24 plants developed the disease and withered in the phage-untreated group, and only three plants were healthy. On the other hand, in the RKP180-treated group, withering was limited to 8 of the 24 plants, and 16 plants were healthy. That is, the number of healthy plants was increased to approximate five times by the RKP180 treatment, with the control value of 62, and thus the control effect of RKP180 was observed.

### Example 7

### Preparation of Bacterial Wilt Disease Control Agent:

The bacteriophage RKP180 separated in Example 1 was adjusted to potency of 3×10⁹ pfu/mL, and a bacterial wilt disease control agent was thus obtained.

### INDUSTRIAL APPLICABILITY

The bacteriophage of the invention can be used for controlling bacterial wilt.

1-6. (canceled)

7: A bacteriophage comprising a gene encoding lysozyme fused to DarB.

8: The bacteriophage of claim 7, comprising genes encoding three lytic enzymes including endolysin, the above-mentioned lysozyme fused to DarB, and Rz/Rz1.

9: The bacteriophage of claim 7, further comprising genes encoding Rz/Rz1, CsrA, and two tail fiber proteins.

10: The bacteriophage of claim 7 that further comprises a gene encoding a tail fiber protein(s) having a receptor binding domain related to the host recognition/binding at the C-terminus.

11: The bacteriophage of claim 7 that further comprises a linear genome or a circular permuted structure.

12: bacteriophage of claim 7 that is RKP180 (NITE BP-03185).

13: The bacteriophage of claim 7, which belongs to family Podoviridae of order Caudovirales of Group I.

14: The bacteriophage of claim 7, which has a head diameter in a range of from 40 to 90 nm, a tail length in a range of from 5 to 30 nm, and a width in a range of from 5 to 20 nm, has a double-stranded genome, has a genome size in a range of from 6,000 to 280,000 bp, has a GC content in a range of from 55 to 75% and comprises from 10 to 330 genes, wherein genomic DNA of the bacteriophage is digested into five or more fragments by restriction enzyme HindIII.

15: The bacteriophage of claim 7 that infects Ralstonia solanacearum.

16: The bacteriophage of claim 7, wherein the Ralstonia solanacearum is strain MAFF107624, strain MAFF211266, strain MAFF211270, strain MAFF211543, strain MAFF301859, strain MAFF311644, strain MAFF730103, strain MAFF730131, strain MAFF302745, strain MAFF311632, strain MAFF211536, strain MAFF331041, strain MAFF730139, strain MAFF211280, strain MAFF211533, strain MAFF211468, strain MAFF211516, strain MAFF311101, strain MAFF311102, strain MAFF211479, strain MAFF211471, strain MAFF211483, strain MAFF211484, strain MAFF211486, strain MAFF211272, strain MAFF211276, strain MAFF211278, strain MAFF211490, strain MAFF211492, strain MAFF211497, strain MAFF211476, strain MAFF211414, strain MAFF211429 and strain MAFF301558.

17. The bacteriophage of claim 7 that is an engineered bacteriophage produced by modifying or mutating genomic DNA of a bacteriophage to contain a gene encoding lysozyme fused to DarB.

18: A composition comprising the bacteriophage of claim 7 and a pharmacologically and botanically acceptable protein stabilizer.

19: The composition of claim 18 which prevents wilt by Ralstonia solanacearum when applied to a plant.

20: The composition of claim 18, wherein the bacteriophage is present at a concentration ranging from 10³ to 10¹² pfu/mL.

21: A method for treating or preventing bacterial wilt disease comprising contacting or injecting into a plant or plant growth medium with the bacteriophage of claim 7.

22: The method of claim 21, wherein the bacteriophage is an engineered bacteriophage produced by modifying or mutating genomic DNA of a bacteriophage to contain a gene encoding lysozyme fused to DarB.

23: The method of claim 21, wherein the plant is infected by Ralstonia solanacearum.

24: The method of claim 21, wherein the plant is infected by Ralstonia solanacearum strain MAFF107624, strain MAFF211266, strain MAFF211270, strain MAFF211543, strain MAFF301859, strain MAFF311644, strain MAFF730103, strain MAFF730131, strain MAFF302745, strain MAFF311632, strain MAFF211536, strain MAFF331041, strain MAFF730139, strain MAFF211280, strain MAFF211533, strain MAFF211468, strain MAFF211516, strain MAFF311101, strain MAFF311102, strain MAFF211479, strain MAFF211471, strain MAFF211483, strain MAFF211484, strain MAFF211486, strain MAFF211272, strain MAFF211276, strain MAFF211278, strain MAFF211490, strain MAFF211492, strain MAFF211497, strain MAFF211476, strain MAFF211414, strain MAFF211429 and strain MAFF301558.

25: The method of claim 21, wherein the plant is a member of Solanaceae, Lamiaceae or Zingiberaceae.

26: The method of claim 21, wherein the plant selected from the group consisting of tomatoes, eggplants, bell peppers, or tobaccos of the family Solanaceae; Perilla frutescens var. crispa or Perilla frutescens of the family Lamiaceae; and ginger, turmeric and curcuma of the family Zingiberaceae.
